(19) United States
(12) Patent Application Publication
(10) Pub. No.: US 2007/0010484 A1
(43) Pub. Date: Jan. 11, 2007

(54) Method for treating or inhibiting the effects of injuries or diseases that result in neuronal degeneration and method for promoting neurogenesis

(75) Inventors: Michal Eisenbach- Schwartz, Rehovot (IL); Ofer Lider, Kfar Bilu Bet (IL); Asya Rolls, Rehovot (IL); Liora Cahalon, Givataim (IL); Osnat Lider, Kfar Bilu B. (IL)

Correspondence Address: BROWDY AND NEIMARK, P.L.L.C.;624 NINTH STREET, NW, SUITE 300, WASHINGTON, DC, 20001-5303, US

(73) Assignee: YEDA RESEARCH AND DEVELOPMENT CO. LTD., Rehovot (IL)

(21) Appl. No.: 11/473,306

(22) Filed: Jun. 23, 2006

Related U.S. Application Data
(63) Continuation-in-part of application No. 10/570,989.
(60) Provisional application No. 60/500,690, filed on Sep. 8, 2003.

(51) Int. Cl. A61K 31/737; A61K 31/728; A61K 31/727

(52) U.S. Cl. 514/54; 514/56

(57) ABSTRACT

Oligosaccharides, and in particular disaccharides, which are degradation products of chondroitin sulfate proteoglycan are effective for use in treating, inhibiting, or ameliorating the effects of injuries or diseases or disorders that result in or are caused by neuronal degeneration or of disorders resulting in mental and cognitive dysfunction. They are also useful for promoting neurogenesis.

### CROSS-REFERENCE TO RELATED APPLICATIONS

This is a continuation-in-part of application No. 10/570,989, which is a 371 national stage application of PCT/US2004/029288, filed Sep. 8, 2004, which claims the benefit of priority to application No. 60/500,690, filed Sep. 8, 2003, the entire contents of which are incorporated herein by reference.

### BACKGROUND OF THE INVENTION

1. Field of the Invention

The present invention relates to methods for promoting neurogenesis and for treating, inhibiting or ameliorating the effects of injuries or diseases that result in neuronal degeneration in the central or peripheral nervous system of a mammal and for promoting recovery from acute CNS injuries or for slowing down degeneration of neurons in chronic neurodegenerative disorders and disorders resulting in mental or cognitive dysfunction.

2. Description of the Related Art

Insults to the central nervous system (CNS) are known to cause widespread degeneration of the affected tissue, often leading to irreversible functional deficits. This devastating outcome results from the primary insult, a self-perpetuating secondary process of damage spread, and the poor ability of damaged neurons to regenerate (Tatagiba, 1997). Studies during the last two decades have focused, among other aspects, on several issues related to recovery after CNS injury, among which are the inhibitory effect of certain CNS-resident compounds on regeneration, emergence of self-destructive compounds such as glutamate at the lesion site (Yoles and Schwartz, 1998; Schwartz, 2003), and the relationship between the local inflammatory response and recovery (Schwartz, 1999; Popovich, 1996), and the inhibitory effect of certain CNS-resident compounds on regeneration (Chen, 2000; Niederost, 2002).

The post-injury extracellular environment of the CNS is characterized by a pronounced expression of chondroitin sulfate proteoglycans (CSPGs), growth-inhibitory matrix protein whose production is up-regulated by several CNS cell types after injury (Morgenstern, 2002). The inhibitory properties of CSPGs have been attributed to their direct inhibitory effect on axonal growth (Fidler P S, 1999; Grimpe B, 2002; McKeon R J, 1995) as well as their pro-inflammatory characteristics (Fitch M T, 1999), and substantiated by the observation that treatment with enzymes which degrade CSPGs results in both growth of axons and attenuation of inflammation (Bradbury E J, 2002; Yick L W, 2000; Zuo J, 2002).

Studies carried out over the last few years, however have provided evidence that a local inflammatory response is part of the body's repair mechanism (Moalem, 1999; Hauben, 2000; Schwartz, 2000; Schwartz, 2001), even if it comes at a price, and that the benefit in the long run outweighs the cost (Hauben, et al., 2000; Moalem, et al., 1999). It was further suggested that although inflammation is frequently observed in degenerating tissues, this process is not necessarily the cause or even a contributory factor in the degeneration. The immune cells that are recruited to a damaged site for therapeutic purposes may simply be insufficiently effective in arresting degeneration or in promoting regeneration, or, alternatively, do not possess the optimal phenotype for facilitating repair (Schwartz, 2001).

The assumption made in the studies that guided the present inventors towards the present invention is that the transient presence of CSPG at the lesion site at an early stage after CNS injury (Jones L L, 2002) might provide an important step in the physiological repair mechanism needed to demarcate the site of the lesion for attracting immune cells to the lesion site in order to stop the spread of damage, albeit at the possible cost of transiently halting neuronal growth (Nevo et al., 2003), and that subsequently, degradation products of CSPG are needed for the ongoing repair. It was shown that in certain tissues other than the CNS, the matrix degradation products play a role in tissue repair (Vaday G G, 2000). No indication for any role of CSPG degradation products or any other degradation products of other matrices in promoting CNS repair has been reported.

Neurocan and phosphacan are two of many chondroitin sulfate proteoglycans that have been described in the brain and were shown to be inhibitors of neurite outgrowth (see, for example, U.S. Pat. No. 5,625,040). U.S. Pat. No. 5,605,938 discloses the use of dextran sulfate and different anionic polymers such as dermatan sulfate, heparan sulfate, chondroitin sulfate, and keratan sulfate in inhibiting neural cell adhesion, migration and neurite outgrowth. U.S. Pat. No. 5,605,891 describes the resumption of neurogenesis process in neuroblastoma cells and of dopamine and noradrenaline concentrations in a rat model of selective sympathetic nervous system lesioning by various glycosaminoglycans. Among the glycosaminoglycans disclosed in U.S. Pat. No. 5,605,891 are heparin, chondroitin 4 sulfate, dermatan sulfate, and a mixture of glycosaminoglycans. U.S. Pat. No. 5,605,891 claims methods of treating acute peripheral neuropathies in a patient using such glycosaminoglycans.

U.S. Pat. No. 6,143,730 discloses sulfated synthetic and naturally occurring oligosaccharides consisting of from three to eight monosaccharide units, which are shown to exert anti-angiogenic, anti-metastatic and anti-inflammatory activities. Among the naturally occurring oligosaccharides tested are chondroitin sulfate tetra-, hexa-, and octasaccharides, the anti-angiogenesis of which was found to be lower than that of other oligosaccharides such as maltotetraose sulfate or maltohexaose sulfate.

U.S. Pat. No. 5,908,837 teaches the use of low doses of low molecular weight heparins (LMWH) in inhibiting inflammatory reactions such as delayed type hypersensitivity (DTH) or the autoimmune disease, adjuvant arthritis, in an animal model. U.S. Pat. No. 6,020,323 further teaches the use of short carboxylated and/or sulfated oligosaccharides, particularly of sulfated disaccharides, in inhibiting inflammatory reactions such as DTH and skin graft rejection, as well as in suppressing autoimmune diseases such as adjuvant arthritis and insulin-dependent diabetes mellitus (IDDM) in NOD mice.

In most brain regions of highly developed mammals, the majority of neurogenesis is terminated soon after birth. However, new neurons are continually generated throughout life in the subventricular zone and the dentate gyrus of the hippocampus. The newly formed neurons originate from neuronal progenitor cells which are found in specific CNS areas and can give rise to all neural lineages: neurons, astrocytes and oligodendrocytes. Insulin-like growth factor 1 (IGF-I) is a polypeptide hormone that has demonstrated effects on these progenitor cells. IGF-I induces proliferation of isolated progenitors in culture, as well as affecting various aspects of neuronal induction and maturation. Moreover, systemic infusion of IGF-I increases both proliferation and neurogenesis in the adult rat hippocampus, and uptake of serum IGF-I by the brain parenchyma mediates the increase in neurogenesis induced by exercise. Neurogenesis in the adult brain is regulated by many factors including aging, chronic stress, depression and brain injury. Aging is associated with reductions in both hippocampal neurogenesis and IGF-I levels, and administration of IGF-I to old rats increases neurogenesis and reverses cognitive impairments. Similarly, stress and depression also inhibit neurogenesis, possibly via the associated reductions in serotonin or increases in circulating glucocorticoids. As both of these changes have the potential to down regulate IGF-I production by neural cells, stress may inhibit neurogenesis indirectly via downregulation of IGF-I. In contrast, brain injury stimulates neurogenesis, and is associated with upregulation of IGF-I in the brain. Thus, there is a tight correlation between IGF-I and neurogenesis in the adult brain under different conditions (Anderson et al. 2002).

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a method for promoting neurogenesis or for treating, inhibiting, or ameliorating the effects of injuries or diseases that result in neuronal degeneration or the effects of disorders that result in mental or cognitive dysfunction, which involves administering to a patient an effective amount of at least one oligosaccharide, which is preferably a degradation product of a naturally-occurring proteoglycan. Alternatively, the method may administer to a patient in need thereof by implantation at the site of neuronal degeneration activated microglial cells, stem cells or neuronal progenitor cells which have been treated with an effective amount of at least one oligosaccharide.

### BRIEF DESCRIPTION OF THE DRAWINGS

FIGS. 1A-1D show that CSPG-derived disaccharides induce axonal growth and prevent growth arrest with FIG. 1A being the control. Incubation of differentiated PC12 cells for 20 min with LPA (1 μg/ml) results in neurite retraction (FIG. 1B). Addition of CSPG-DSs (5 or 50 μg/ml) together with LPA resulted in dose-dependent reversal of the retraction process (FIGS. 1C. and 1D).

FIGS. 2A and 2B are graphs showing the assessment of neurite length on PC12 cells. The longest neurite on each cell was measured and the average length of the longest neurites was expressed as a percentage of the average length of the longest neurites in the control group (FIG. 2A). In FIG. 2B, the percentage of cells bearing neurites longer than 10 μm is expressed as mean±SEM. * P<0.05, ** P<0.005, *** P<0.0005; scale bar: 50 μm.

FIG. 3 is a graph showing that CSPG-derived disaccharides induce neurite outgrowth in NGF-differentiated PC12 cells. PC12 cells were left untreated or were incubated for 3 days with NGF (10 ng/ml) and sulfated or non-sulfated DS. Cells were fixed with 4% PFA and analyzed by light microscopy. Values represent the total length (mean±SEM) of neurites per cell; *P<0.05, **P<0.005, ***P<0.0005. Representative data from one of seven experiments are shown.

FIGS. 4A-4C show that CSPG-derived disaccharides prevent neural cell death. Rat OHSCs were incubated with CSPG-DSs for 24 h. They were then labeled with propidium iodide and examined under a fluorescence microscope, where FIG. 4A is the control (untreated) OHSCs compared to OHSCs that were incubated with 50 μg/ml of CSPG-DS (FIG. 4B). The intensity of propidium iodide staining in the treated groups, expressed as a percentage of the intensity in the control group (mean±SD is shown in FIG. 4C). *P<0.05, **P<0.005. Representative data from one of four experiments are shown.

FIG. 5 is a graph showing that CSPG-derived disaccharides promote neuronal survival in a model of glutamate toxicity injected into the eye. C57Bl/6J mice were injected intravitreally with a toxic dose of glutamate (200 nmol). Immediately thereafter, the mice were divided into two groups. Mice in one group were left untreated and those in the other group were injected i.v. with the sulfated CSPG-DSs. Mice in a third group were not subjected to glutamate toxicity and received only CSPG-DSs. The number of surviving RGCs was assessed 1 week later, and is expressed as a percentage (mean±SEM) of the number of surviving RGS in the group of rats not subjected to glutamate toxicity (n=6 mice per group). Representative data from one of two experiments are shown.

FIG. 6 is a graph showing that CSPG-DS reduces pathological symptoms of experimental autoimmune encephalomyelitis in mice. C57/black mice were immunized with an encephalitogenic peptide of MOG to induce EAE symptoms (day 0). The mice were then divided into four groups (n=6 per group), each injected i.p. with 5 μg of CSPG-DS in different regimen: mice in the first group were injected only on day 0, those in the second group were injected on days 0 and 7, those in the third group were injected on days 0, 3, 5, and 7, and those in the fourth group (control) remained untreated. The EAE score was determined as described in Materials and Methods section.

FIG. 7 is a graph showing that CSPG-DS protects rats against experimental autoimmune uveitis. Lewis rats were immunized with R16 emulsified in CFA. On days 3, 6, 9, 12, and 17 after immunization each rat received an i.p. injection of 15 μg of CSPG-DS (n=6) or 15 μg of MP (n=6) or no treatment (n=6). RGC survival was measured in terms of the mean number of RGCs retrogradely labeled with rhodamine dextran 3 weeks after immunization, expressed as a percentage of the mean number of surviving RGCs in normal eyes (P***<0.0005).

FIG. 8 is a graph showing that CSPG-DS inhibits the delayed-type hypersensitivity response in mice. After induction of DTH, the mice were divided into five groups (n=4 per group) and were either left untreated or injected with CSPG-DS at the concentrations indicated in the figure. The DTH response was assessed by measuring swelling of the ears. Changes in sizes of the swelling of the ear are expressed as the percentage of inhibition relative to the untreated group. The results of one representative experiment out of three are shown. (*, P<0.05; ***, P<0.0005.)

FIGS. 9A-9C show that CSPG-DS affects T-cell motility and activates the suppressors of cytokine signaling protein. Human T cells were isolated from healthy blood donors and labeled with ⁵¹[Cr]. The cells were then preincubated for 2 h at the indicated concentrations of CSPG-DS. For analysis of T-cell migration, the cells were washed and placed in the upper chamber of a transwell apparatus. SDF-1α was introduced into the lower chamber. Migration of T cells through FN-coated filters into the lower chamber was assayed after 3 h by measuring the radioactivity in the lower chamber. Values are expressed as percentages of control. The results of one representative experiment out of three are shown in FIG. 9A. To assay T-cell adhesion, the T cells that were preincubated with CSPG-DS- were replated on FN-coated microtiter plates in the presence of SDF-1α. After 1 h nonadherent cells were washed off, the bound cells were lysed, and the radioactivity of the lysates was measured. Values are expressed as percentages of control. The results of one representative experiment out of three are shown in FIG. 9B. T cells were incubated in the presence of CSPG-DS at the indicated concentrations for 3 h, then lysed, and the lysates were analyzed on SDS-gels. Total PYK2 antibody was used as a control for measurement of total protein. The results of one representative experiment out of four are shown in FIG. 9C.

FIGS. 10A-10D show that CSPG-DS affects cytokine secretion from human T cells. Human T cells were preincubated with CSPG-DS at the indicated concentrations for 2 h, then replated on 24-well plates precoated with anti-human CD3 antibody. After 24 h, the supernatants were collected and the amounts of secreted IFN-γ (FIG. 10A) and TNF-α (FIG. 10B) were determined by ELISA. The data are means (±SD) of five experiments. NF-κB that translocated to the nuclei was assayed by lysing the nuclear extracts of human T cells, treated as described above, to determine IFN-γ and TNF-α secretion from those cells. As a control for total protein in the nuclei, β-lamin was used. One represetnative experiment out of three is shown in FIG. 10C. To determine the mRNA levels of human T cells that were pretreated with CSPG-DS (2 h, in the indicated concentration) and activated with a CD3 for 12 h, total mRNA was extracted from those cells and assayed for IL-4 or IL-13 by RT-PCR. As a control we used the GAPDH gene. The results of one representative experiment our of four are shown in FIG. 10D.

FIG. 11 is a graph showing administration of CSPG-DS in chronic IOP rat model reduces death rates of RGCs. Intravenous administration of CSPG-DS (15 μg per injection) was given in two different regimens: on the seventh day after the first laser irradiation and every other day starting on day 7 to day 14 after the first laser session. The effective regimen was when CSPG-DS was given every other day (p<0.0001 when compared to the PBS injected group).

FIG. 12 is a graph showing topical administration of CSPG-DS proves effective in protecting RGCs from chronic IOP induced death. Using the same rat model of chronic IOP elevation topical administration (as eye drops) of CSPG-DS (a concentration of 20 μg/ml was added at 50 μl drops every 5 minutes for a total of 5 drops in 25 minutes) was performed every other day starting from the seventh day after first laser irradiation and ending on day 14. On day 21, retinas were labeled and viable RGCs incorporated the dye and were counted. After flat mounding of the retina under flurescence microscope (×800). Significantly higher numbers of viable RGCs per mm² were noted in the CSPG-DS treated animals (n=6) when compared to the PBS treated ones (n=4) p<0.0001.

FIG. 13 is a graph showing that disaccharides from different sources promote neural survival in PC12 cell cultures Increase in cell survival after treatment of PC12 cell cultures with disaccharides, expressed as a percentage (mean±SD)of the survival of cells not treated with disaccharides, determined by XTT assay (n=4). Cell death was induced in PC12 cell cultures by a toxic dose of glutamate (10⁻³ M). Representative data from one of two experiments are shown (* p<0.05, relative to control PC12 cells without disaccharides).

FIG. 14 shows fluorescence micrographs for detecting IGF-1 expression in microglial cultures. CSPG-DS was added in the indicated concentration (25 μg/ml or 50 μg/ml) to microglia cultures derived from C57bl/6j new born mice. An hour later, LSP was added in the indicated concentration (10 ng/ml, 25 ng/ml, or 50 ng/ml) for additional 24 hours. Cells were labeled with anti-IFG-1 antibody and detected by fluorescent microscopy.

### DETAILED DESCRIPTION OF THE INVENTION

Chondroitin sulfate proteoglycan (CSPG) is transiently elevated following traumatic spinal cord injury. Several works have attributed to it a negative role in post-traumatic recovery due to its inhibitory effect on axonal growth and its pro-inflammatory properties, viewing inflammation as detrimental to neuronal survival. In Example 1 presented herein below, the present inventors demonstrate that CSPG disaccharides (CSPG-DSs) can activate microglia to express MHC II, a marker of activated microglia phenotype associated with tissue repair. The disaccharide (DS) degradation products of CSPG were found by the present inventors to enhance neuronal survival in vivo after exposure to glutamate toxicity, to promote neurite outgrowth in vitro, to retain the ability to induce MHC II expression in microglial cells and to promote neurogenesis.

CSPG and its derived DSs are believed to play a key role in CNS repair, possibly by first demarcating the damaged site and thereby isolating the still-healthy tissue from the damaged neurons. Subsequently, the disaccharide degradation products of CSPG can control/modulate the local immune response and promote neuronal repair. Intervention with DSs is a strategy for CNS repair, representing a way of boosting the physiological repair process.

The present invention provides a method for treating, inhibiting, or ameliorating the effects of injuries or diseases that result in neuronal degeneration or the effects of disorders that result in mental or cognitive dysfunction and a method for promoting neurogenesis. These methods involve administering to a patient in need thereof an effective amount of at least one oligosaccharide, such as degradation products of a naturally-occurring proteoglycan (PG), e.g., chondroitin sulfate proteoglycan (CSPG), which the present inventors discovered have the ability to (i) maintain the CSPG effect of activating microglia to induce MHCII expression and acquire a phenotype associated with tissue repair, (ii) promote neurite outgrowth and neurogenesis, and (iii) allow better survival of stressed neurons. Alternatively, the at least one oligosaccharide is used to treat stem cells or neuronal progenitor cells prior to the cells being administered to the patient by implantation at the site of neuronal degeneration. The method of the present invention which promotes neurogenesis is involved in cell renewal in the CNS, and includes all types of CNS cells. Thus, it may involve neurogenesis, astrogenesis, oligodendrogenesis, microgliagenesis, as well as cell renewal or “genesis” of other CNS cell types.

An embodiment of the present invention is used to inhibit secondary degeneration which may otherwise follow primary NS injury, e.g., closed head injuries and blunt trauma, such as those caused by participation in dangerous sports, penetrating trauma, such as gunshot wounds, hemorrhagic stroke, ischemic stroke, glaucoma, cerebral ischemia, or damages caused by surgery such as tumor excision, or may even promote nerve regeneration in order to enhance or accelerate the healing of such injuries or of neurodegenerative diseases such as those discussed below. In addition, the method may be used to treat, inhibit, or ameliorate the effects of disease or disorder that result in a degenerative process, e.g., degeneration occurring in either gray or white matter (or both) as a result of various diseases or disorders of the central or peripheral nervous system, including, without limitation: diabetic neuropathy, senile dementias, Alzheimer's disease, Parkinson's Disease, facial nerve (Bell's) palsy, glaucoma, Huntington's chorea, amyotrophic lateral sclerosis (ALS), status epilepticus, non-arteritic optic neuropathy, intervertebral disc herniation, vitamin deficiency, prion diseases such as Creutzfeldt-Jakob disease, carpal tunnel syndrome, peripheral nerve injuries and peripheral and localized neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease, lipoproteinemia, autoimmune diseases such as multiple sclerosis, etc. In light of the findings with respect to the glutamate protective aspect of the present invention, other clinical conditions that may be treated in accordance with the present invention include epilepsy, amnesia, anxiety, hyperalgesia, psychosis, seizures, abnormally elevated intraocular pressure, oxidative stress, and opiate tolerance and dependence. In addition, the glutamate protective aspect of the present invention, i.e., treating injury or disease caused or exacerbated by glutamate toxicity, can include post-operative treatments such as for tumor removal from the CNS and other forms of surgery on the CNS. Included in the disorders treated, inhibited or ameliorated by the present invention are those chronic neurodegenerative disorders and disorders resulting in mental or cognitive dysfunction such as memory loss. Conditions associated with memory loss and cognitive dysfunction include aging.

Oligosaccharides, and in particular disaccharides, derived from naturally-occurring proteoglycans are preferably the degradation products of the glycosaminoglycan (GAG) chain found in proteoglycans. While chondroitin sulfate proteoglycan (CSPG), heparan sulfate proteoglycan (HSPG), dermatan sulfate proteoglycan (DSPG), hyaluronic acid (HA), and keratan sulfate proteoglycan (KSPG) are the preferred proteoglycans from which the oligosaccharides are derived, with HSPG more preferred and CSPG most preferred, there are other proteoglycans that may be suitable.

Proteoglycans are abundant in nature. The following is a list of non-limiting examples of proteoglycans, some of which are only partly proteoglycans but have the common feature that they all contain the GAG moiety/chain: decorin, biglycan, fibromodulin, lumican, PRELP, keratocan, osteoadherin, epiphycan/proteoglycan Lb, osteoglycin/mimecan, oculoglycan, opticin, asporin, aggrecan, versican, neurocan, brevican, collagens, serglycins, syndecans, betaglycan, phosphatidyl inositol-anchored proteoglycans, CD44 proteoglycan family, thrombomodulin, invariant g chain, perlecan, agrin, bamacan, phosphacan, NG2 proteoglycan, and miscellaneous neuronal proteoglycans. Versican, decorin, biglycan, and aggrecan bind a chondroitin sulfate moiety, whereas CD44 binds either chondroitin sulfate or heparin sulfate GAG moieties. Some modifications and variations of the GAG moieties may be found in proteoglycans. Using HSPG as an example, heparan sulfate chains exhibit remarkable structural diversity. Although heparan sulfate chains are initially synthesized as a simple alternating repeat of glucuronosyl and N-acetylglucosaminyl residues joined by β1-4 and α1-4 linkages, there are many subsequent modifications. The polysaccharide is N-deacetylated and N-sulfated and subsequently undergoes C5 epimerization of glucuronosyl units to iduronosyl units, and various O-sulfations of the uronosyl and glucosaminyl residues. The variability of these modifications allows for some thirty different disaccharide sequences which, when arranged in different orders along the heparan sulfate chain, can theoretically result in a huge number of different heparan sulfate structures. In this regard, the anticoagulant polysaccharide heparin, present only in mast cell granules, represents an extreme form of heparan sulfate where epimerization and sulfation have been maximized. Most heparan sulfates contain short stretches of highly sulfated residues joined by relatively long stretches of non-sulfated units. Preferably, the naturally-occurring proteoglycan used in the present invention is a human proteoglycan.

It is also preferred that the oligosaccharides used in the present invention be enzymatic degradation products of naturally-occurring proteoglycans such as CSPG, although other means of degrading naturally-occurring proteoglycans to oligosaccharides, preferably to disaccharides, such as by reaction with nitric oxide (nitric oxide products degrade chondroitin sulfate; Nitric Oxide 2(5):360-356, 1998), by chemical depolymerization, i.e., by nitrous acid, by β-elimination, or by periodate oxidation, may be suitable as well. The conditions of depolymerization can be carefully controlled to yield products of desired molecular weights. Such oligosaccharide degradation products of naturally-occurring proteoglycans, such as CSPG-DS, can also be prepared synthetically rather than be generated by degradation directly from a naturally-occurring proteoglycan. It will be appreciated by those of skill in the art that further synthetic modifications can be made to the oligosaccharide.

With regard to enzymatic degradation, the oligosaccharides used in the method of the present invention are preferably obtained by degradation of glycosaminoglycan with a glycosaminoglycan degrading enzyme that naturally degrades that particular glycosaminoglycan in vivo in the body of a mammal. Non-limiting examples of such enzymes that can degrade glycosaminoglycan include matrix metalloproteinases (e.g., MMP-2, MMP-3, MMP-8, MMP-9, MMP-12, MMP-15, etc.; Ferguson et al., 2000), plasmin, thrombin, and hyaluronidase. A review of extracellular matrix (ECM) and cell surface proteolysis is presented by Werb (1997). Other enzymes, such as chondroitinase ABC, AC, B, or C (Du et al., 2002 and Saito et al., 1968; Volpi, 2000; Huang et al., 1995), heparinase I, II, or III, and keratinase isolated from bacteria (and commercially available from Sigma, St. Louis, Mo.), for example, can be suitably used to obtain disaccharides in vitro for use in the present invention.

The oligosaccharide, and in particular the disaccharide, degradation products of proteoglycans can be obtained by a series of chromatographic purification steps. An initial purification may be made using a low pressure size-exclusion gel chromatography (i.e., Sephadex columns) followed by high pressure liquid chromatography (HPLC). The purification scheme to isolate and purify oligosaccharides may use, for example, gel permeation HPLC or strong anion exchange (SAX) HPLC columns. Methods for the detection of disaccharides formed as degradation products of chondroitin sulfate have been reported (Huang et al., 1995; Volpi, 2000). Similarly, an analytical method for determining the disaccharide degradation products of chondroitin sulfate, as well as of other proteoglycans, such as dermatan sulfate and hyaluronic acid, by the action of degradative enzymes has been developed (Sugahara et al., 1996).

Non-limiting examples of sulfated disaccharides from chondroitin sulfate are: 2-acetamido-2-deoxy-3-O-(β-D-gluco-4-enepyranosyluronic acid)-4-O-sulfo-D-galactose, also known as α-4-deoxy-L-threo-hex-4-enopyranosyluronic acid-[1→3]-N-acetyl-D-galactosamine-4-sulfate (Di-4S; Sigma catalog no. C4045); 2-acetoamido-2-deoxy-3-O-(β-D-gluco-4-enepyranosyluronic acid)-6-O-sulfo-D-galactose, also known as α-4-deoxy-L-threo-hex-4-enopyranosyluronic acid-[1→3]-N-acetyl-D-galactosamine-6-sulfate (Di-6S; Sigma catalog no. C4170); β-glucuronic acid-[1→3]-N-acetyl-D-galactosamine-6-sulfate (Δi-6S; Sigma catalog no. C5945); and α-4-deoxy-L-threo-hex-4-enopyranosyluronic acid-2-sulfate-[1→3]-N-acetyl-D-galactosamine (Di-UA-2S; Sigma catalog no. C5820). A non-limiting example of non-sulfated disaccharide from chondroitin is 2-acetamido-2-deoxy-3-O-(β-D-gluco-4-enepyranosyluronic acid)-D-galactose, also known as a-4-deoxy-L-threo-hex-4-enopyranosyluronic acid-[1→3]-N-acetyl-D-galactosamine (Di-OS; Sigma catalog no. C3920).

Preferably, the disaccharide is sulfated. More preferably, the disaccharide is Di-6S.

Non-limiting examples of disaccharides from heparin sulfate, a form of heparan sulfate, are: α-4-deoxy-L-threo-hex-4-enopyranosyluronic acid-2-sulfo-[1→4]-D-glucosamine-6-sulfate (Sigma catalog no. H8892); α-4-deoxy-L-threo-hex-4-enopyranosyluronic acid-[1→4]-D-glucosamine-6-sulfate (Sigma catalog no. H9017); α-4-deoxy-L-threo-hex-4-enopyranosyluronic acid-2-sulfo-[1→4]-D-glucosamine (Sigma catalog no. H9142); α-4-deoxy-L-threo-hex-4-enopyranosyluronic acid-[1→4]-D-glucosamine acetate (Sigma catalog no. H0895); α-4-deoxy-L-threo-hex-4-enopyranosyluronic acid- [1→4]-D-glucosamine (Sigma catalog no. H9276); heparin disaccharide I-P (Sigma catalog no. H9401); heparin disaccharide I-S (Sigma catalog no. H9267); heparin disaccharide II-S (Sigma catalog no. H1020); heparin disaccharide III-S (Sigma catalog no. H9392); and heparin disaccharide IV-S (Sigma catalog no. H1145).

While it is preferred that the oligosaccharide is a disaccharide derived from CSPG as a product of CSPG degradation, other oligosaccharides which produce the desired result, i.e., capable of treating, inhibiting or ameliorating the effects of injury or disease that results in neuronal degeneration or capable of promoting neurite outgrowth, can suitably be used in the method of the present invention. Such oligosaccharides may be naturally occurring oligosaccharides or may be synthetic, although it is preferred that the oligosaccharide be a sulfated oligosaccharide. The oligosaccharide may be a tri-, tetra-, penta-, hexa-, hepta-, octasaccharide, etc., and may contain only one type of monosaccharide unit or may contain more than one type of monosaccharide units. Besides being derivatized by a sulfate moiety, as in the preferred sulfated oligosaccharide or disaccharide embodiment, the monosaccharide units of the oligosaccharide may be derivatized with phosphate, acetyl or other moieties.

The oligosaccharide(s) which is used in the method of the present invention may be administered alone, or in combination with other therapies. For example, the oligosaccharide(s) may be efficaciously combined with a cytokine, lymphokine, growth factor, or colony-stimulating factor, in the treatment of neurodegenerative diseases. Exemplary cytokines, lymphokines, growth factors, and colony-stimulating factors for use in combination with the oligosaccharide(s) include, without limitation, EGF, FGF, interleukins 1 through 12, M-CSF, G-CSF, GM-CSF, stem cell factor, erythropoietin, and the like. In addition, the oligosaccharide(s) may be combined with such neurotrophic factors as CNTF, LIF, IL-6 and insulin-like growth factors.

The oligosaccharide used in accordance with the present invention may be formulated in a pharmaceutical composition in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

The term “carrier” refers to a diluent, adjuvant, excipient, or vehicle with which the therapeutic is administered. The carriers in the pharmaceutical composition may comprise a binder, such as microcrystalline cellulose, polyvinylpyrrolidone (polyvidone or povidone), gum tragacanth, gelatin, starch, lactose or lactose monochydrate; a disintegrating agent, such as alginic acid, maize starch and the like; a lubricant or surfactant, such as magnesium stearate, or sodium lauryl sulfate; a glidant, such as colloidal silicon dioxide; a sweetening agent, such as sucrose or saccharin; and/or a flavoring agent, such as peppermint, methyl salicylate, or orange flavoring.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local (i.e., locally administered at the site of injury or neuronal damage).

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in rectal compositions such as suppositories or retention enemas, e.g., containing conventional suppository bases such as cocoa butter or other glycerides.

Furthermore, the compositions may be formulated for local administration to the eyes such as in the form of eye drops.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

The oligosaccharide used in the methods of the present invention may be formulated in accordance with routine procedures as pharmaceutical compositions adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

When the oligosaccharide is to be introduced orally, it may be mixed with other food forms and consumed in solid, semi-solid, suspension, or emulsion form; and it may be mixed with pharmaceutically acceptable carriers, including water, suspending agents, emulsifying agents, flavor enhancers, and the like. In one embodiment, the oral composition is enterically-coated. Use of enteric coatings is well known in the art. For example, Lehman (1971) teaches enteric coatings such as Eudragit S and Eudragit L, The Handbook of Pharmaceutical Excipients, 2^{nd} Ed., also teaches Eudragit S and Eudragit L applications. One Eudragit which may be used in the present invention is L30D55.

The oligosaccharide may also be administered nasally in certain of the above-mentioned forms by inhalation or nose drops. Furthermore, oral inhalation may be employed to deliver the disaccharide to the mucosal linings of the trachea and bronchial passages.

The oligosaccharide used in the methods of the present invention is preferably administered to a mammal, preferably a human, shortly after injury or detection of a degenerative lesion in the nervous system.

The oligosaccharide(s) is administered in a manner compatible with the dosage formulation, and in a therapeutically effective amount. A therapeutic amount of the oligosaccharide(s) is an amount sufficient to produce the desired result, e.g., to treat, inhibit or ameliorate the effects of injury, disease or disorder that results in neuronal degeneration, to promote neurite outgrowth, etc. In the case of in vivo therapies, an effective amount can be measured by improvements in neuronal regeneration, to name one example. The administration can vary widely depending upon the disease condition and the potency of the therapeutic compound. The quantity to be administered depends on the subject to be treated, the capacity of the subject's system to utilize the active ingredient, and the degree of therapeutic effect desired. Precise amounts of active ingredient required to be administered depend on the judgment of the practitioner and are peculiar to each individual. Thus, the dosage ranges for the administration of the oligosaccharide are those large enough to produce the desired effect in which the symptoms of disease, e.g., neuronal degeneration--are ameliorated or decreased. The dosage should not be so large as to cause adverse side effects, although none are presently known. Generally, the dosage will vary with the age, condition, and sex of the patient, as well as with the extent and severity of the disease in the patient, and can be determined by one of skill in the art. The dosage can be adjusted by the individual physician in the event of any complication.

Effective amounts of the oligosaccharide(s) may be measured by improvements in neuronal or ganglion cell survival, axonal regrowth, and connectivity following axotomy (see, e.g., Bray, et al., (1991)). Improvements in neuronal regeneration in the CNS and PNS are also indicators of the effectiveness of treatment with the disclosed compounds and compositions, as are improvements in nerve fiber regeneration following traumatic lesions (Cadelli, et al., 1992; Schwab, 1991).

The oligosaccharide may be administered as a single dose or may be repeated. The course of treatment may last several months, several years or occasionally also through the life-time of the individual, depending on the condition or disease which is being treated. In the case of a CNS injury, the treatment may range between several days to months or even years, until the condition has stabilized and there is no or only a limited risk of development of secondary degeneration. In chronic human disease or Parkinson's disease, the therapeutic treatment in accordance with the invention may be for life.

As will be evident to those skilled in the art, the therapeutic effect depends at times on the injury or disease to be treated, on the individual's age and health condition, on other physical parameters (e.g., gender, weight, etc.) of the individual, as well as on various other factors, e.g., whether the individual is taking other drugs, etc.

Having now generally described the invention, the same will be more readily understood through reference to the following example which is provided by way of illustration and is not intended to be limiting of the present invention.

### EXAMPLE 1

### Disaccharides Derived From Chondroitin Sulfate Proteoglycans Overcome Growth Arrest and Neurotoxicity

Chondroitin sulfate proteoglycans (CSPGs) inhibit central nervous system (CNS) axonal regeneration (Morgenstern D A, 2002), and their local degradation promotes recovery (Bradbury E J, 2002; Yick L W, 2000). The assumptions underlying the present study were that the increased expression of CSPGs observed after injury is part of the self-repair mechanism needed for transient demarcation of the lesion site (Nevo, 2003), and that their degradation products subsequently participate in the cascade leading to neuronal repair. Here, the present inventors show that CSPG-derived disaccharides (DSs), the major building blocks of CSPGs, participate in the rescue of neurons from the consequences of mechanical injury ex vivo and from glutamate-induced neurotoxicity in vivo. Moreover, CSPG-DSs induced neurite outgrowth and prevented neurite collapse (via a Rho-dependent pathway) induced by lysophosphatidic acid in cultured PC12 cells. CSPG-DSs might provide a means of circumventing a common extracellular signal for death or growth arrest imposed by various environmental elements, including intact CSPGs, and other growth inhibitors. The present inventors believe that exogenous supply of CSPG-DSs might therefore be a way to promote repair after acute CNS injuries or in chronic neurodegenerative conditions.

Materials and Methods

Reagents: The following reagents and chemicals were purchased from the sources indicated: fetal calf serum (FCS), horse serum, fetal bovine serum, HEPES buffer, antibiotics, sodium pyruvate, and Dulbecco's modified Eagle's medium (DMEM) were from Beit-Ha-Emek, (Kibbutz Beit Ha-Emek, Israel). NGF, polyoxyethylene sorbitan monolaurate (TWEEN 20), phosphate-buffered saline (PBS), ascorbic acid, L-glutamate and LPA were from Sigma (St. Louis, Mo.). The non-sulfated sodium salt (Di-0S) and the sulfated sodium salt (Di-6S) of CSPG-derived disaccharides (DSs) were purchased from Sigma (Steinheim, Germany). Collagen was from Calbiochem-Novabiochem, (Darmstadt, Germany).

Animals: C57B1/6J mice were supplied by the Animal Breeding Center of the Weizmann Institute of Science. All animals were handled according to NIH guidelines for the management of laboratory animals and they were housed in a light and temperature-controlled room and matched for age in each experiment.

PC12 cell line: Rat pheochromocytoma (PC12) cells were cultured in DMEM containing 8% horse serum and either 8% FCS (culture medium) or 1% FCS (differentiation medium), and antibiotics. For assays of neurite outgrowth, the cells were plated (10⁵ cell/well) on 13-mm glass coverslips, precoated with collagen (500 μg/ml) in 24-well plates.

Treatment of PC12 cells with lysophosphatidic acid (LPA). PC12 cells were placed and were differentiated for 3 days in the presence of 100 ng/ml NGF, in collagen-precoated culture dishes (Corning). The differentiated cells were left untreated or were treated for 20 min with 1 μg/ml LPA, either alone or together with 50 μg/ml CSPG-DSs. The cells were then fixed with 4% paraformaldehyde (PFA) and analyzed by Nomarski microscopy. The longest neurite of each cell was measured and the results are expressed as their mean±SEM.

Neurite outgrowth assays: PC12 cells were cultured for 24-72 h while being stimulated with 10 ng/ml NGF, with or without CSPG-DSs. Cell morphology was visualized under a phase-contrast microscope and neurite lengths were measured using ImagePro. At least 200 cells were measured for each condition.

Glutamate-induced toxicity: C57Bl/6J mice were anesthetized by intraperitoneal (i.p.) injection of ketamine (80 mg/kg; Fort Dodge Laboratories, Fort Dodge, Iowa) and xylazine (16 mg/kg; Vitamed, Israel). Their right eyes were punctured with a 27 gauge needle in the upper part of the sclera and a hamilton syringe with a 30-gauge needle was inserted as far as the vitreal body. Each mouse was injected with a total volume of 1 μl saline containing L-glutamate (100 nmol). Mice in one group were also injected intravenously (i.v.) with 5 μg of CSPG-DSs in 200 μl saline (Schori, 2001).

Labeling of retinal ganglion cells: Mice were anesthetized as described above and placed in a stereotactic device. The skull was exposed and the bregma identified and marked. The site selected for injection was in the superior colliculus, 2.92 mm posterior to the bregma, 0.5 mm lateral to the midline, at a depth of 2 mm from the brain surface (Franklin and Paxinos, 1997). A window was drilled in the scalp above the designated coordinates in the right and left hemispheres. The neurotracer dye FluoroGold (5% solution in saline; Fluorochrome, Denver, Colo.), was stereotactically applied (1 μl, at a rate of 0.5 μl/min in each hemisphere) using a Hamilton syringe, and the skin over the wound was sutured. After 72 h, the mice were killed with a lethal dose of pentobarbitone (170 mg/kg), their eyes were enucleated, and retinas were detached from the eyes and prepared as flattened whole mounts in 4% PFA in PBS.

Assessment of retinal ganglion cell survival: Retinas were examined for labeled retinal ganglion cells (RGCs) by fluorescence microscopy. Labeled cells from four to six fields of identical size (0.076 mm²) were counted. The selected fields were located at approximately the same distance from the optic disk (0.3 mm) to counteract variations in RGC density as a function of distance from the optic disk. Cells were counted under the fluorescence microscope (magnification ×800) by observers blinded to the treatment received by the mice. The average number of RGCs per field was calculated for each retina. The number of RGCs in the contralateral (uninjured) eye was also counted and served as an internal control.

Organotypic hippocampal slice cultures (OHSC): OHSC were prepared as described (Franklin and Paxinos, 1997) from rats brains (Lewis aged 8-10 days) CSPG-DSs were added to the OHSCs for 24 h. Propidium iodide (5 μg/ml; Sigma) was then added to the medium for 30 min. The brain slices were examined under a Zeiss laser-scanning confocal microscope (LSM510) or a Zeiss Axioplane 100 fluorescence light microscope.

Results and Discussion

Disaccharides Derived From Chondroitin Sulfate Proteoglycan Protect Neurons Against Growth Arrest

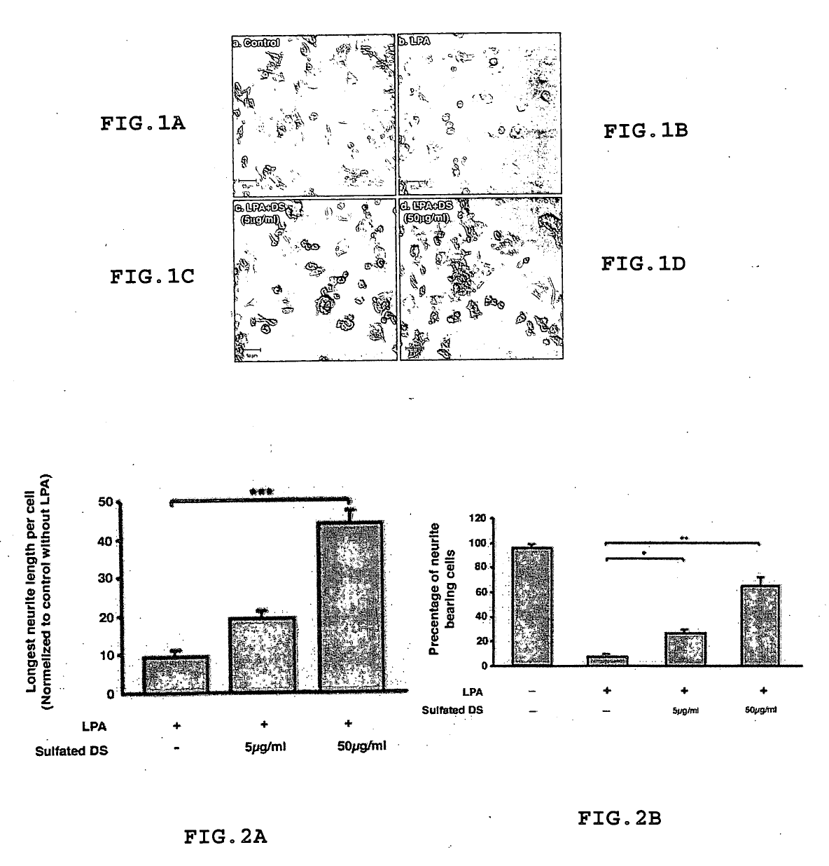

CSPG-DSs constitute the building blocks of CSPGS. They include Di-6S (a sulfated DS, which possesses a sulfate group O-linked at position 6 on the galactosamine unit), and the non-sulfated Di-0S. First, the ability of the sulfated CSPG-DS to protect neurons from growth arrest was examined. PC12 neuronal cells were cultured, with or without the addition of CSPG-DSs, in the presence of nerve growth factor (NGF) and in the presence or absence of LPA, an axon-collapsing agentknown to activate a Rho-dependent pathway. LPA by itself, as expected (Tigyi G, 1996), induced neurite collapse (FIG. 1B). This collapse was prevented, however, when LPA was applied together with CSPG-DSs (FIGS. 1C and 1D). The addition of CSPG-DSs had a beneficial effect on the number of neurite-bearing cells and on the mean neurite length (FIGS. 2A and 2B). Since the axonal collapse caused by CSPGs, or by other growth-arresting compounds including LPA, is reportedly mediated via signal transduction pathways in which Rho plays a central role (Kranenburg O, 1999), these findings suggest that the beneficial effect of the CSPG-DSs is Rho-associated.

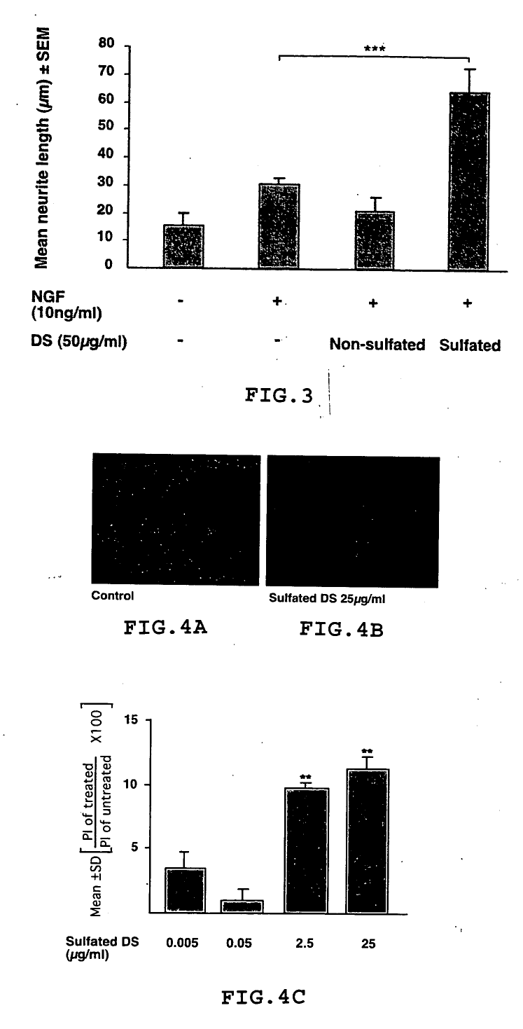

Next, CSPG-DSs were examined as to whether they can contribute to neurite growth and extension. The effect of the sulfated CSPG-DSs on neurons was examined in PC12 cells in the presence of a low concentration of NGF (10 ng/ml). The mean length±SD of neurites in PC12 cells cultured on collagen in the presence of NGF was 14.7±4 μm. When CSPG-DSs were added to the cultures, the mean neurite length was increased to 107±7.8 μm (FIG. 3). Non-sulfated Di-0S had no effect on neurite outgrowth. Thus, the sulfated DSs derived from CSPGs not only rescue neurites but also induce neurite outgrowth.

DSs Derived From CSPGs Protect Neural Tissue Against Mechanical Injury and Glutamate Toxicity

Organotypic hippocampal slice cultures (OHSCs) are used to study ex vivo the effects of different treatments on the protection or destruction of neurons after a primary CNS injury. Excision of these slices from the intact brain simulates a mechanical injury to the hippocampal tissue, and the subsequent loss of neurons simulates post-traumatic secondary degeneration. Immediately after sectioning the rat brain, hippocampal slices were incubated in the presence or absence of CSPG-DSs. FIGS. 4A and 4B show OHSCs stained with propidium iodide (indicating cell death). Exposure of OHSCs to CSPG-DSs (2.5 μg/ml or 25 μg/ml) significantly reduced neuronal loss (FIGS. 4B and 4C).

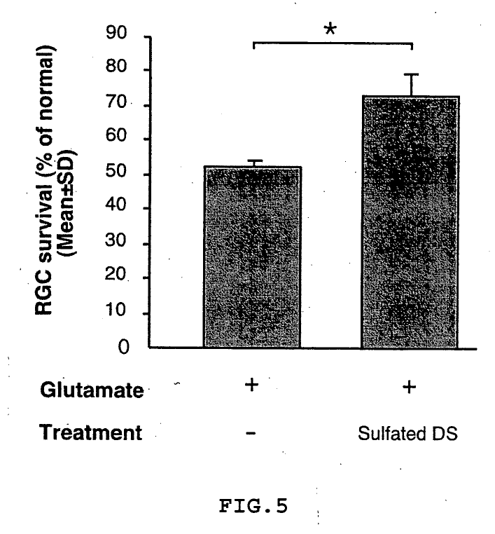

These findings prompted the present inventors to examine the ability of CSPG-DSs to protect neurons subjected to neurotoxicity in vivo. The model of choice was glutamate intoxication, since the presence of glutamate in toxic amounts is a common finding in both acute and chronic degenerative conditions of the CNS. Retinal ganglion cells (RGCs) of mice were exposed to a toxic dose of intravitreally injected glutamate. Since DSs are low-molecular-weight compounds (approximately 600 daltons), they were administered systemically. RGC survival was assessed after the mice were treated with sulfated CSPG-DSs administered intravenously (i.v.) by a single injection. The number of surviving RGCs per mm2 (mean±SEM) was 1404±56 in the absence of CSPG-DSs and 1965±166 after CSPG-DSs treatment (FIG. 5). Given that the total number of RGCs per mm2 counted under the same experimental conditions in normal retinas is 2200±203 (mean±SEM), treatment with CSPG-DSs caused a significant increase (P<0.05) in the ability of neurons to overcome threatening conditions. A similar protective effect against glutamate toxicity was observed when treatment with CSPG-DSs was administered intravitreally (data not shown). Intravitreal injection of CSPG-DSs in the absence of glutamate had no effect on neuronal survival.

These findings thus show that the disaccharidic products of CSPG degradation, and specifically Di-6S, play a key role in CNS repair by circumventing neuronal growth arrest apparently via a Rho-dependent pathway, stimulating neurite outgrowth in vitro, and protecting against glutamate intoxication in vivo.

A number of authors have reported an increase in the extracellular matrix-associated CSPGs at an early stage after CNS injury, with marked effects on both inflammation and growth inhibition (Fitch M T, 1997; Fidler P S, 1999; Grimpe B, 2002; McKeon R J, 1995). All of those authors assumed that the post-traumatic presence of CSPGs is detrimental for recovery. It is conceivable, however, that the presence of CSPGs in the early stages after injury is a critical requirement for isolating the site of lesion and stopping the spread of damage (Nevo, 2003). Studies over the last 5 years have shown that a well-regulated and properly synchronized healing process requires a well-controlled local inflammatory reaction, in which the healing-related activities of resident microglia are triggered by helper T cells (Moalem, 2000; Schwartz, 2003). According to this view, the beneficial effect observed after spinal cord injury following local application of chondroitinase ABC (Bradbury E J, 2002), a CSPG-degrading enzyme, might be a result of the local generation of specific CSPG-DSs.

Regeneration can be assumed to be a net outcome of the fine balance between the need for survival and the need for regrowth, as well as the intracellular balance between signaling for growth arrest (induced by the environment) and for axonal regrowth. The temporal and spatial requirements of these various needs and components do not necessarily coincide. It is conceivable that once CSPG is degraded, further requirements for survival and regrowth are compromised by the presence of its disaccharidic degradation products. If the injury is severe, the physiological supply of these degradation products might therefore not be adequate, in terms of timing or quantity or both, to counteract the transient growth arrest imposed by CSPGs and other growth inhibitors. In such a case, their exogenous application might have a significant therapeutic effect, by promoting axonal elongation even while the neuronal environment is one of growth arrest (e.g., it contains intact CSPGs). Moreover, the finding that exogenous application of CSPG-derived DSs is beneficial for axonal growth and neuronal survival suggests that CSPG degradation is important not necessarily because it eliminates the intact molecule, but because it yields DSs. The production of soluble DSs might provide a way to circumvent a common extracellular signal for death or growth arrest imposed by various Rho-activating environmental elements, including intact CSPGs, NOGO, and other myelin-associated growth inhibitors (Niederost B, 2002; Monnier P P, 2003). In studies demonstrating axonal collapse, this phenomenon has usually been associated with activation of Rho. It therefore seems likely that the CSPG-DSs rescue neurons and that they do this via a Rho-dependent pathway. Activation of Rho can lead not only to growth arrest but also to axonal elongation, depending on the recruitment of additional signaling molecules that participate in the transition from inhibition to stimulation of neurite outgrowth (Arakawa Y, 2003). The transition requires an appropriate balance between Rho and Rac-based signaling pathways (Dickson, 2001) and possibly also involves additional pathways yet to be identified.

The fact that the signals from the CSPG-DSs are the opposite of those emitted by the intact CSPGs might be explained if two assumptions are made: firstly, that the same receptor mediates both the interaction of neurons with CSPGs and their interaction with the CSPG-DSs, and secondly, that in the former case, because of the multivalency of the DS-binding sites on the intact molecule, the mediation occurs via a cross-linked type of receptor signaling pathway, whereas the interaction with a single DS activates a monovalent signaling cascade.

The observed CSPG-DS-induced protection of neurons from glutamate intoxication suggests that the CSPG-DSs, in addition to their effect on neurons, affect the behavior of microglia in a way that helps the latter to buffer glutamate toxicity (Schwartz M, 2003). Studies have shown that in order to help protect the tissue against glutamate toxicity the local innate immune response must be controlled, and that this can be achieved by suitable activation of microglia, for example by delivering T cells to the lesion site (Schori, 2001; Schori, 2002; Schwartz M, 2003). To be effective, these T cells must be specific to antigens presented at the site of glutamate toxicity (Mizrahi T., 2002). Once properly activated, the microglia acquire a phenotype that allows them to clear the lesion site of glutamate toxicity and other potentially harmful factors. It is possible that CSPG-DSs directly activate the microglia to acquire the necessary phenotype.

### EXAMPLE 2

### Disaccharides Derived From Chondroitin Sulfate as a Treatment for Inflammation-Mediated Neurodegeneration

Chondroitin sulfate proteoglycan (CSPG) represents a diverse class of complex macromolecules that share a general molecular structure, comprising a central core protein with a number of covalently attached carbohydrate chains, the glycosaminoglycans (GAGs). Each GAG is made up of repeating disaccharide (DS) units (glucuronic acid/iduronic acid-N-acetylgalactosamine), which are either not sulfated or possess one sulfate per DS (Hascall et al., 1970).

Studies both in vivo and ex vivo have demonstrated that CSPG is a major growth inhibitor in the central nervous system (CNS), however the inhibitory mechanisms are not clear; inhibition by CSPG might be receptor-mediated (Dou et al., 1997 and Ernst et al., 1995), or might result from the molecule's biophysical or biochemical characteristics (Dillon et al., 2000; Morris, 1979; Zuo et al., 1998; and Condic et al., 1999). CSPG is prominently expressed during CNS development (Wilson et al., 2000; Kitagawa et al., 1997 and Meyer-Puttlitz et al., 1996) and directs neuronal growth by preventing the spread of axons to growth-restricted areas (Fukuda et al. 1997). In the adult brain its expression is down-regulated (Kitagawa et al., 1997), but is increased after traumatic injuries to the CNS (Morgenstern et al., 2002; Lemons et al., 1999; Lips et al., 1995; McKeon et al., 1999; and Properzi et al., 2003), mainly at the margins of the lesion site (Jones et al., 2002; Matsui et al., 2002; and Tang et al., 2003). Elevated expression of CSPG has also been reported in other CNS disorders, such as in sites of β-amyloid aggregation (DeWitt et al., 1996) and in the active plaques seen in multiple sclerosis (MS) (Sobel et al., 2001). It is interesting to note that CSPG expression occurs in several types of CNS injuries independently of the nature of the primary insult and it might therefore suggest on a role for this molecule in a physiological mechanism of repair. However, numerous studies have shown that after an injury, improved repair and better recovery result from CSPG degradation (Yick et al., 2000; Bradbury et al., 2002; Zuo et al., 2002; Tropea et al., 2003; and Chau et al., 2004). In a previous study, the present inventors were able to reconcile these apparently conflicting observations by showing that CSPG serves as part of the repair mechanism when the intensity and the timing of its activity are suitably controlled; when not well regulated, however, CSPG appears to contribute to the pathology. Moreover CSPG degradation, as the present inventors have previously shown, yields reparative compounds contributing to CNS repair (Example 1; Rolls et al., 2004).

Neurodegenerative disorders can result from a number of different factors, including immunopathologic injuries. Thus, as much as the immune cells are needed for repair, malfunctioning of the local immune system can lead to neurodegeneration in the CNS. Yet, it is becoming clear that a local immune response is needed for maintenance of the CNS both in non-pathological conditions and also has an important role to fight off various CNS pathologies regardless of whether their cause is immunological (as in the case of autoimmune diseases) or non-immunological (such as Alzheimer's and Parkinson's diseases and glaucoma). Since the common factor in all of these diseases is the need for a controlled local immune response that does not endanger neurons, in the present study the possibility that the disaccharidic breakdown products of CSPG, which were recently shown to exert a beneficial effect on microglial activation and on neuronal survival (Example 1; Rolls et al., 2004), might serve the dual role of controlling the activity of the systemic T cell mediated response and activating the local immune cells, the microglia, to exert a neuroprotective response was examined.

Materials and Methods

Reagents. FCS, horse serum, FBS, HEPES buffer, antibiotics, sodium pyruvate, and DMEM were all purchased from Beit-Ha-Emek, Kibbutz Beit Ha-Emek, Israel. Phosphatase inhibitor cocktail, PBS, β-mercaptoethanol, RPMI-1640, and BSA were from Sigma-Aldrich, St. Louis, Mo. Other reagents used were the sodium salt (CSPG-DS) of chondroitin sulfate disaccharides (C-4170) (Sigma, Steinheim,Germany); fibronectin (FN; Chemicon, Temecula, Calif.); stromal-cell-derived factor-la (SDF-1α and and recombinant human SDF-1α (R&D Systems, Minneapolis, Minn.); and Na₂⁵¹[Cr]O₄ (Amersham Pharmacia Biotech, Little Chalfont, UK).

Animals. C57Bl/6J and Balb/c mice and Lewis rats were supplied by the Animal Breeding Center of The Weizmann Institute of Science. All animals were handled according to NIH Guidelines for the Management of Laboratory Animals. They were housed in a light- and temperature-controlled room and were matched for age in each experiment.

Human T cells. T cells from the peripheral blood of healthy donors were isolated by negative selection using a RosetteSep™ antibody cocktail containing mabs against CD16, CD19, CD36, and CD56 (StemCell Technologies, Vancouver, BC). After incubation with the cocktail for 20 min at room temperature, blood samples were diluted in PBS with 2% fetal bovine serum, loaded on a Ficoll column (ICN Biomedical, Aurora, Ohio), and centrifuged at 1200× g for 20 min at room temperature. The cells were removed from the Ficoll column, washed, and cultured in RPMI containing antibiotics and 10% heat-inactivated FCS.

Induction of experimental autoimmune encephalomyelitis. Mice were immunized s.c. at one site in the flank with 200 μl of emulsion consisting of myelin oligodendrocyte glycoprotein (MOG) 1-22 (300 μg per mouse) emulsified in CFA supplemented with 500 μg of Mycobacterium tuberculosis (Difco, Detroit, Mich.). Clinical symptoms of experimental autoimmune encephalomyelitis (EAE) were examined and scored daily, as follows: 0, no clinical disease; 0.5, piloerection; 1, tail weakness; 1.5, tail paralysis; 2, hindlimb weakness; 3, hindlimb paralysis; 3.5, forelimb weakness; 4, forelimb paralysis; 5, moribund state or death.

Induction of experimental autoimmune uveitis. To induce experimental autoimmune uveitis (EAU), Lewis rats were immunized with R16 (30 μg), a peptide derived from an ocular antigen IRBP emulsified in CFA containing 2.5 mg/ml M. tuberculosis. A total volume of 100 μl was injected s.c. into each rat at the root of the tail. Rats were then divided into three groups. On days 3, 6, 9, 12 and 17 after immunization the rats in the first group were injected i.p. with CSPG-DS (15 μg/rat), and rats in the second group were injected i.p. with methylprednisolone (MP. 30 mg/kg; Solu-Medrol, 125 mg/ml, Pharmacia & Upjohn, Puurs, Belgium). Rats in third group were left untreated.

Assay for delayed-type hypersensitivity. Groups of female inbred Balb/c mice (n=4 per group) were sensitized with 2% oxazalone (100 μl;) dissolved in acetone/olive oil (4:1 (vol/vol)) applied topically on the shaved abdominal skin. A delayed-type hypersensitivity (DTH) response was elicited 5 days later by challenge with 0.5% oxazalone in acetone/olive oil (10 μl applied topically to each side of one ear, and measured with an engineer's micrometer (Mitutoyo, Elk Grove Village, Ill., Tokyo, Japan)). Immediately before and 24 h after antigen challenge, the marked area was measured again.

T-cell adhesion assays. Adhesion of T cells to FN was assayed as described (Ariel et al., 1998). Briefly, flat-bottomed microtiter well plates were precoated with CSPG or FN (10 μg/ml) and the remaining binding sites were blocked with 1% BSA. ⁵²[Cr]-labeled T cells were resuspended in RPMI medium supplemented with 1% HEPES buffer and 0.1% BSA (adhesion medium). After preincubation 2 h with CSPG-DS at the indicated concentrations, the T cells were incubated (30 min, 37° C., humidified atmosphere of 7% CO₂ in air) with SDF-1α and then added to the wells. The contents of the wells were further incubated (30 min, 37° C., humidified atmosphere of 7% CO₂ in air) and then gently washed. Adherent cells were lysed with lysis buffer (1 M NaOH, 0.1% Triton X-100 in H₂O), removed, and counted with a γ-counter (Packard, Downers Grove, Ill.).

T-cell chemotaxis. Migration of purified human T cells was measured with a transwell apparatus (6.5 mm diameter; Corning, New York, N.Y.) fitted with polycarbonate filters (pore size 5 μm). The filters separating the upper and lower chambers were pretreated with FN (20 μg/ml) for 1 h at 37° C. ⁵¹[Cr]-labeled T cells were preincubated for 2 h with CSPG-DS at the indicated concentrations, and then suspended (2×10⁶/ml) in RPMI containing 0.1% BSA, 0.1% L-glutamine, and antibiotics, and added to the upper chamber. The bottom chambers contained the same RPMI medium, with or without human SDF-1α (50 ng/ml). After 3 h of incubation at 37° C. and a humidified atmosphere of 7% CO₂ in air, the migration of T cells through the coated filters was assayed by collecting the transmigrated cells from the lower chambers, lysing them in lysis buffer, and counting them with a γ-counter.

Assay of IFN-γ secretion. Human T cells were purified and maintained in culture (RPMI containing 10% FCS, 1% pyruvate, 1% glutamine, 1% antibiotics, in a humidified atmosphere of 7% CO₂in air), and the cells were activated for 2 h with the indicated concentrations of CSPG-DS. In order to stimulate the cells to secrete cytokines, they were replated (1×10⁶ cells in 0.5 ml culture medium per well) in 24-well plates precoated with 1 μg/ml immobilized anti-CD3 mAb (non-tissue-culture grade). After 24 h the supernatants were collected and their IFN-γ contents determined by ELISA, using anti-IFN-γ mAb (Pharmingen, San Diego, Calif.) according to the manufacturer's instructions.

Western blot analysis of T-cell nuclear extracts. Purified T cells (5×10⁶) were preincubated for 2 h with different concentrations of CSPG-DS. The cells were then replated at the same CSPG-DS concentration on 24-well plates pre-coated for 24 h with anti-CD3 mAb. The T cells were lysed in 10 mM HEPES, 1.5 mM MgCl₂, 1 mM dithiothreitol, 1 mM PMSF, and 0.5% Nonidet P-40 (buffer A). The lysates were incubated on ice for 10 min and centrifuged at 2000 rpm for 10 min at 4° C. The supernatants (cytoplasmic extracts) were transferred and the pellets (nuclei) were suspended in buffer containing 30 mM HEPES, 450 mM NaCl, 25% glycerol, 0.5 mM EDTA, 6 mM dithiothreitol, 12 mM MgCl₂ 1 mM PMSF, 10 μg/ml leupeptin, 10 μg/ml pepstatin, and 1% phosphatase inhibitor cocktail (buffer B), and the suspension was incubated on ice for 30 min. The lysates were cleared by centrifugation (30 min, 14×10³ rpm, 4° C.), and the resulting supernatants were analyzed for protein content. Sample buffer was added, the mixture was boiled, and the samples containing equal amounts of proteins were separated on 10% SDS-PAGE gel and transferred to nitrocellulose membranes. The membranes were blocked with TBST buffer containing low-fat milk (5%), Tris pH 7.5 (20 mM), NaCl (135 mM), and Tween 20 (0.1%)), and probed with the following mabs, all diluted 1:1000 in the same buffer: anti-NF-κB, anti-suppressors of cytokine signaling protein (anti-SOCS-3), anti-total PYK2 and anti-laminin B. Antibodies were purchased from Santa Cruz Biotech (Santa Cruz, Calif.). Immunoreactive protein bands were visualized using labeled secondary antibodies and the enhanced chemiluminescence system. For assay of SOCS-3, the cells were incubated for 3 h with CSPG-DS, cell lysis was performed without separating the nuclei from the cytoplasm (so that the cells were lysed only with buffer B), and the procedure was completed as described above.

RNA purification, RT-PCR, and CDNA synthesis. After pretreatment with the indicated concentrations of CSPG-DS for 2 h, the T cells were replated for 12 h on 24-well plates pre-coated with anti-CD3 mAb, lysed with TRI reagent (MRC, Cincinnati, Ohio), and total cellular RNA was purified from lysates using the RNeasy kit (Qiagen, Hilden, Germany) according to the manufacturer's instructions. RNA was converted to cDNA using SuperScript II (Promega, Madison, Wis.), as recommended by the manufacturer. The expression of specific mRNAs was assayed by RT-PCR, using the messageScreen™ Human Th1/Th2 Cytokine Set 1 Multiplex PCR® kits (BioSource International, Camarillo, Calif.), according to the manufacturer's instructions.

T-cell apoptosis: T cells (2×10⁶ cells/ml) were incubated for 2 h with the indicated concentrations of CSPG-DS in RPMI medium containing 10% FCS, and then plated on 24-well plates (non-tissue-culture grade) precoated with anti-CD3 mAb (1 μg/ml; overnight) and cultured for 72 h. The percentage of cells undergoing apoptosis was determined using the annexin V-detection assay (Bender MedSystem, San Bruno, Calif.). The cells were incubated for 10 min in the dark at room temperature in 200 μl of buffer containing FITC-conjugated human annexin V (5 ml; Bender MedSystem, Propidium iodide (10 μl) was added to each sample, and the percentage of cells undergoing apoptosis was analyzed by FACS® at 525 nm using CELLQuest Software. Cells that stained positively for annexin V and negatively for propidium iodide corresponded to the apoptotic cells.

Statistical analysis: Statistical analysis was performed using Student's t-test.

Results

CSPG-DS Alleviates Experimental Autoimmune Encephalomyelitis in Mice

EAE is an autoimmune inflammatory disease used as an animal model for MS (Lublin, 1985). In susceptible mouse strains, EAE can be induced by active immunization with CNS proteins or peptides such as myelin basic protein, proteolipid protein, or MOG, all emulsified in adjuvant, or by the passive transfer of T cells reactive to such CNS antigens. In both MS and EAE, Th1 cytokines in the CNS at the peak of disease are present in abundance. A number of studies indicate that the pathogenesis of EAE is mediated by myelin-specific Th1 cells that secrete IFN-γ, TNF-α, and IL-2 (Olsson, 1995).

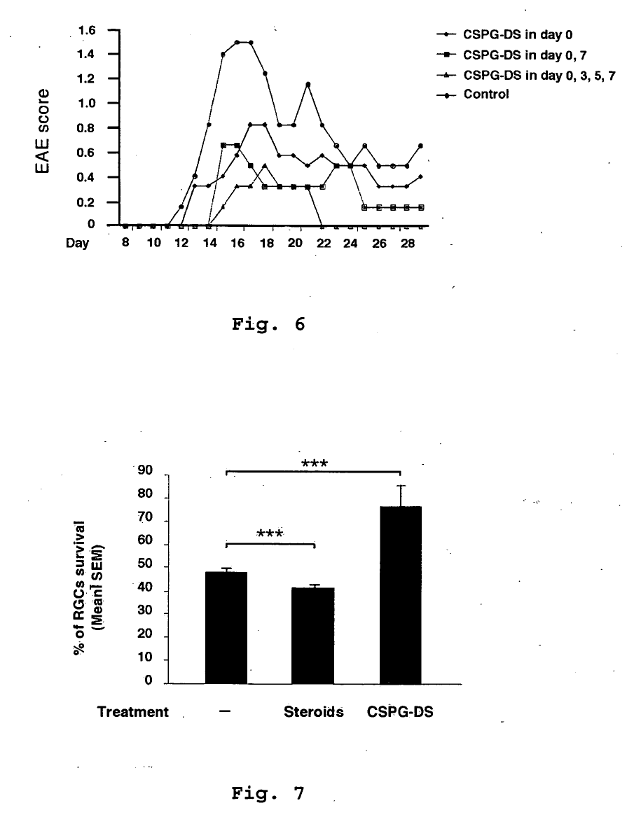

In the present study, EAE was induced in four groups of mice. To examine the effect of CSPG-DS on the course of the disease, the mice in three groups were injected with i.v. CSPG-DS according to different regimens: mice in the first group were injected only on day 0, mice in the second group on day 0 and day 7, and mice in the third group on days 0, 3, 5, and 7. Mice in the fourth group (control) were injected only with PBS. FIG. 6 shows a dose-dependent decline in severity of the induced disease with increasing frequency of CSPG-DS injection. The repeated injections of CSPG-DS on days 0, 3, 5, and 7 significantly attenuated the symptoms of the disease, and shortened its duration. However, less frequent injections could also alleviated the disease.

CSPG-DS Protects RGCs From Experimental Autoimmune Uveitis

An immune response is the body's defense against threatening situations, even if the threat derives from the immune system itself. Accordingly, the present inventors postulated that the best way to overcome immunopathological injuries to the CNS is not by eliminating the immune response (which is the rationale underlying treatment with steroids), but rather by modulating it.

To test this hypothesis, EAU was induced in Lewis rats. EAU is a classical model for immunopathological injury causing neuronal death in the eye (Thurau et al., 2003). It can be induced by passive transfer of T cells directed against ocular antigens, such as interphotoreceptor retinal-binding protein (IRBP), or, as in the present study, by active immunization with the antigen itself or with an antigen-derived peptide such as R16, which is derived from IRBP (Caspi, 1999). Immunized rats were treated with a steroid (MP) to eliminate the immune response, or with CSPG-DS. A third group of R16-immunized rats was left untreated. The regimen for steroid treatment was adapted from protocols previously used to treat rats with EAU (Bakalash et al., 2003). By counting the surviving RGCs in each group, immunization of naive Lewis rats with R16 emulsified in CFA was shown to cause EAU symptoms that were accompanied by a RGC loss of 52±2% (mean±SD) relative to normal rats (FIG. 7). Treatment of the R16-immunized rats with MP increased the RGC loss to 59±1.6% (mean±SD). In contrast, treatment of the R16-immunized rats with CSPG-DS was neuroprotective, and resulted in a RGC loss of only 24±9% (FIG. 7).

CSPG-DS Attenuates the Delayed-Type Hypersensitivity Response in Mice

The above results raised the question of whether the protective effect of CSPG-DS observed in rats with EAE and EAU reflects the previously demonstrated ability of CSPG-DS to protect neurons from injurious conditions regardless of the primary cause of damage (Example 1 and Rolls et al., 2004) or is it also mediated via regulation of immune factors associated with autoimmune diseases. To address this question, the DTH model, usually applied to analyze the effects of a specific compound on T-cell migration or activation, was used. Activation and recruitment of cells into an area of inflammation are crucial steps in development of the DTH response. Effect of reduction of the DTH response are usually attributed either to the decreased presence of T cells in the irritated regions (meaning reduced T-cell migration) or to a decline in their activity (meaning reduced cytokine secretion) (Kobayashi et al., 2001). FIG. 8 shows that the DTH response in mice treated with CSPG-DS was significantly weaker than in untreated mice (40% reduction in DTH response at the most efficient concentration of 1 μg/ml), indicating that CSPG-DS can affect immune components which can be associated with autoimmune diseases.

CSPG-DS Down-Regulates T-cell Motility

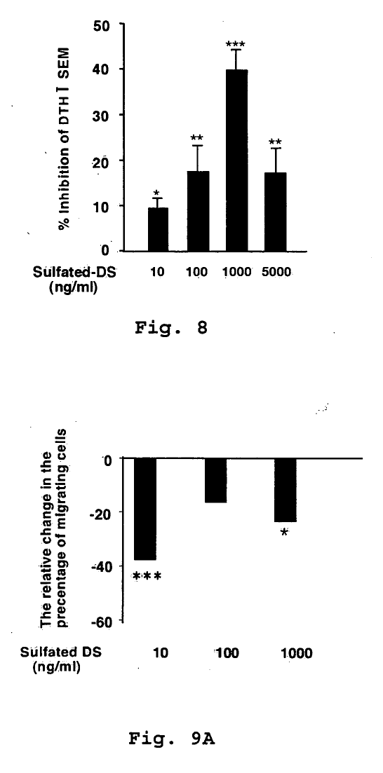

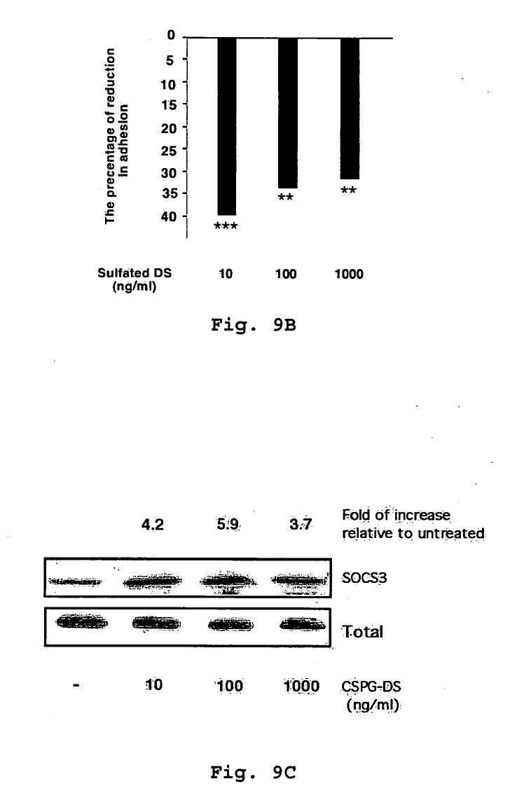

As mentioned above, attenuation of the DTH response is usually correlated with reduced T-cell motility or function, which is related in turn to the secretion of Th1-associated cytokines IFN-γ, and TNF-α. To determine whether the effect of CSPG-DS is mediated via a direct effect on T-cell motility, the migration of T cells towards a chemoattractive agent, SDF-1α, was assessed in a transwell migration apparatus. SDF-1α is an effective chemoattractant for T cells in the CNS (Moser et al., 1998; Wu et al., 2000) and it is associated with several CNS immunopathological insults (Fang et al., 2004; Pashenkov et al., 2003). After treatment of T cells with CSPG-DS for 2 h, their migration towards SDF-1α in the transwell migration apparatus was reduced relative to that of untreated cells (FIG. 9A).

A prerequisite for T-cell migration is the adhesion of T cells to a matrix or target cell. Such adhesion typically arrests the normal flow of the T cells, allowing them to migrate to their destination. In an attempt to understand how CSPG-DS reduces T-cell migration we examined its effect on T-cell adhesion to SDF-1α, known to induce the activation and promote the adhesion of T cells (Fang et al., 2004; Pashenkov et al., 2003) was examined. The adhesion of T cells that were pretreated with CSPG-DS for 2 h prior to their exposure to SDF-1α was significantly reduced relative to that of untreated T cells (FIG. 9B).

T-cell growth, differentiation, and chemotactic responses require coordinated action between cytokines and chemokines and their intracellular targets. The present inventors were therefore interested in determining whether CSPG-DS can also affect an intracellular mechanism known to be associated with an attenuated response to chemokines. The SOCS-3 family of proteins have been identified as feedback regulators of JAK/STAT activation through their binding to JAK kinases or cytokine receptors (Cooney, 2002). Therefore, by down-regulating the chemokine-mediated activation signal, these proteins reduce migration both in vivo and in vitro in several contexts (Soriano et al., 2002). SOCS-3 specifically down-regulates signals associated with responses mediated through the SDF-1a receptor CXCR4 (Soriano et al., 2002). Pretreatment of T cells with CSPG-DS for 2 h resulted in an increase in SOCS-3 relative to untreated T cells (FIG. 9C), suggesting that CSPG-DS suppresses the signaling pathway through which SDF-1α mediates its effects.

CSPG-DS Reduces Secretion of IFN-65 and TNF-α by Activated T Cells

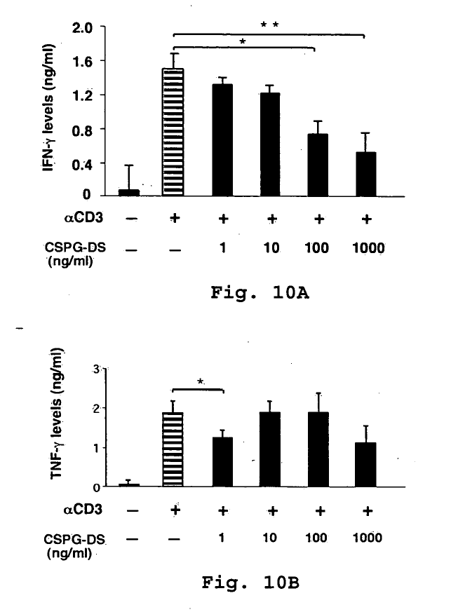

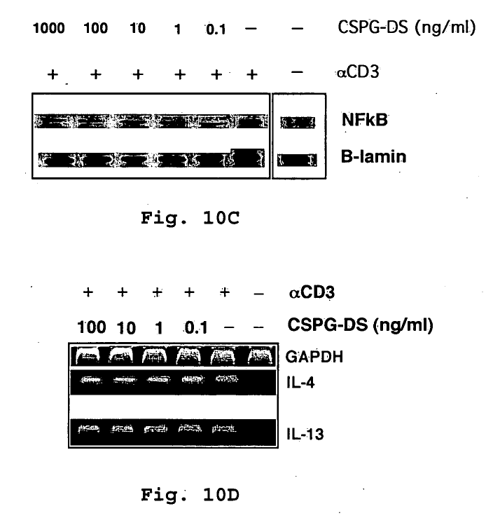

The observed effect of CSPG-DS on the DTH response is generally thought to derive from either reduced motility or decreased function of T cells in terms of secretion of the Th1-associated cytokines IFN-γ, TNF-α, or both. The effects of CSPG-DS on the secretion of cytokines by T cells was therefore examined. Pretreatment of T cells for 2 h with CSPG-DS prior to their activation with anti-CD3 antibodies, simulating physiological stimulation through the TCR, caused a significant reduction in their secretion of IFN-γ (FIG. 9A) and TNF-α (FIG. 10B).

This study shows that CSPG-DS can affect the intracellular mechanism that suppresses the cytokine-signaling pathway. Such suppression can account for many of the observed effects of CSPG-DS in down-regulating T-cell activation and motility. However, the present inventors were interested in finding an intracellular pathway that might reduce the secretion of cytokines directly. A likely candidate might be the NF-κB cascade, a major signaling pathway. The activity of NF-κB is governed by its translocation to the nucleus, where it controls the transcription of genes responsible for regulating cell proliferation, cell survival, and inflammation (Makarov, 2000). The ability of CSPG-DS to regulate NF-κB activity mediated via TCR activation by the anti-CD3 Ab was examined. FIG. 10C shows a reduction in NF-κB levels, thus supporting the possibility that the NF-κB pathway is a mechanism through which CSPG-DS can reduce T-cell activation by down-regulating the secretion of IFNγ and TNF-α.

CSPG-DS Does Not Affect Secretion of Th2-Associated Cytokines

A number of factors shown to down-regulate the secretion of Th1-associated cytokines can also induce a phenotype switch in the cytokine-secretion profile of activated T cells. Moreover, as shown by several authors, attenuation of EAE can be correlated with the secretion by Th-2 cells of the cytokines IL-4 and IL-13, which play a regulatory role that contributes to the recovery. To determine whether the observed CSPG-DS-mediated down-regulation of IFN-γ and TNF-α is also associated with an increase in Th2-associated cytokines, the mRNA levels of of IL-4 and IL-13 in T cells that were pretreated with CSPG-DS for 2 h, washed, and then activated by incubation with anti-CD3 antibody was analyzed. FIG. 10D records the mRNA content of each examined cytokine. Th2-associated cytokines were not affected by the treatment with CSPG-DS. The results shown in the figure are from T cells incubated with anti-CD3 Ab for 3 h; similar results were obtained after incubation for 6 or 12 h.

CSPG-DS Does Not Induce T-Cell Apoptosis

To exclude the possibility that the observed down-regulation of T-cell activation and migration after treatment with CSPG-DS was the result of apoptosis rather than of the change in T-cell activation, the effect of CSPG-DS on T-cell apoptosis was examined. Activation of T cells with anti-CD3 antibody induced T-cell apoptosis and the percentage of cells undergoing apoptosis was determined using the annexin V-detection assay. However, treatment with CSPG-DS did not significantly affect the viability of the T cells.

Discussion

The results of this study showed that CSPG-DS, a product of enzymatic degradation of CSPG, alleviates the clinical symptoms of EAE and EAU in mice. It also down-regulated a DTH response in vivo and reduced T-cell migration and cytokine secretion in vitro. The reduction in T-cell motility could be a result of decreased T-cell adhesion, an important step for the migration process, or an increase in SOCS-3, a suppressor of cytokine signaling, or both. The observed ability of CSPG-DS to reduce the secretion of IFN-γ and TNF-α by anti-CD3-activated T cells might be attributable, at least in part, to its effect on the NF-κB pathway. CSPG-DS did not, however, increase the secretion of Th2-associated cytokines such as IL-4 and IL-13 by the activated T cells, nor did it affect their viability.

The composition of CSPG in the CNS is dynamic and its levels vary during development (Kitagawa et al., 1997; Lemons et al., 1999). It is associated mainly with growth inhibition (Silver et al., 2004), serving an important role in directing axonal growth during development (Silver et al., 2004). After an injury to the CNS, CSPG in the vicinity of the injured site is increased (Morgenstern et al., 2002; Lemons et al., 1999; Lips et al., 1995; McKeon et al., 1999; and Properzi et al., 2003), and it forms a barrier to axonal growth (Silver et al., 2004). This latter property led a number of authors to suggest that degradation of CSPG (by its specific enzyme chondroitinase ABC) is beneficial for CNS regeneration (Yick et al., 2000; Bradbury et al., 2002; Zuo et al., 2002; Tropea et al., 2003; and Chau et al., 2004). The results in Example 1 showed, however, that a product of such enzyme-catalyzed degradation strongly affects both neurons and microglia. The observed correlation between the increase in CSPG following various CNS insults and under various neurodegenerative conditions such as MS (Sobel et al., 2001), Alzheimer's disease (DeWitt et al., 1996), glaucoma (Knepper et al., 1996) and other pathologies, irrespective of the primary cause of damage or in the type of damage inflicted, led the present inventors to believe that CSPG is actually associated with a general, nonselective mechanism of CNS repair. The finding that products of CSPG degradation are highly effective not only in promoting neuronal survival and growth but also in activating the CNS-resident immune cells (microglia) led us to postulate that CSPG-DS might also be effective in modulating an immune response under immunopathological conditions of the CNS by providing a multicellular treatment that protects neurons and modulates immune functions.

To test this hypothesis, mice with EAE and mice with EAU were used as models of CNS damage generated by immune pathology. In both models, the cause of damage is associated with the presence of activated T cells in the CNS (Olsson, 1995; and Thurau et al., 2003). The primary reasons for the induction of the corresponding human disease, although not clear, were suggested to derive from bacterial invasion of the CNS, resulting in loss of control of the immune response (Johnson et al., 1996). CSPG-DS, a disaccharidic breakdown product of CSPG, was effective in alleviating the clinical symptoms in both models. These observations were further supported by the finding here that CSPG-DS could also down-regulate a DTH response known to be mediated, as in the EAE and EAU models, by activated T cells, and in particular by those characterized by secretion of Th1-associated cytokines.

In seeking to further characterize the mechanism through which CSPG-DS alleviates the clinical symptoms of the two experimental diseases: EAE and EAU, the present inventors discovered that CSPG-DS is a potent inhibitor of T-cell activation and migration. It significantly reduced both the adhesion of T cells and their responsiveness to cytokine-mediated signaling, thus reducing their motility. However, although CSPG-DS down-regulated Th1-associated cytokine secretion, it did not induce a phenotypic change in the T cells, nor did it affect the production of Th2-associated cytokines. This observation is in line with previous studies indicating that prevention of MS does not require a phenotype switch, and that control of IFN-γ and TNF-α concentrations might be sufficient (Betteli et al., 2004). It is also in line with the previous finding in the laboratory of the present inventors that the very same T cells which are destructive in MS are protective in the context of the injury, provided that their amounts and the cytokines they produce are controlled (Moalem et al., 1999). The observed reduction in IFN-γ and TNF-α can be attributed, at least to some extent, to the decrease in NFκB caused by CSPG-DS. NF-κB plays a critical role in the regulation of immunity and inflammation by stimulating the transcription of many cytokine genes, including TNF-α and IFN-γ (Ghosh et al., 1998), however, the assays of apoptosis showed that CSPG-DS did not cause T-cell death, and therefore can not provide an explanation for the effects in cytokines levels.

The immune system is the part of the organism responsible for fighting off any threat to its health. It therefore seems reasonable to assume that such conditions include immune-mediated neuropathology even though the cause of damage in such cases is related to an imbalance in the immune response. However, complete suppression of the immune response (as demonstrated, for example, by the use of steroids to treat EAU in the present study), failed to improve disease outcome. The present inventors therefore suggest that that modulation rather than suppression of the immune response, by providing a multicellular treatment for immunopathological injuries of the CNS, is likely to yield more effective repair of the damaged tissue. Such modulation was manifested in the present study, by the effect of CSPG-DS in reducing the intensity of the T-cell mediated response, and in a previous study in which the laboratory of the present inventors used CSPG-DS to activate microglia towards a neuroprotective phenotype. The ability of CSPG-DS to activate the microglia to a neuroprotective phenotype, while at the same time removing harmful T cells from the CNS, suggests that this breakdown product is a promising candidate for the treatment of immune-mediated neuropathological conditions.

### EXAMPLE 3

### Chondroitin Sulfate Proteoglycan-Derived Disaccharides as a Therapeutic Compound for Glaucoma

In the eye, CSPG is highly abundant, serving many functional roles during development and maintenance of the tissue (Koga et al., 2003). For example, it was shown that CSPG contributes to the stromal transparency in the corneal tissues and also contributes to neuronal network formation and maintenance of the interphotoreceptor matrix (Tanihara et al., 2002). CSPG is further upregulated in pathological condition of the eye such as in glaucoma (Tezel et al., 1999; Johnson et al., 1996). It was directly shown in histochemical studies that CSPG levels are elevated in cases of laser-induced glaucoma and antibodies against CSPG were observed in patients with glaucoma (Tezel et al., 1999; Johnson et al., 1996).

In the last years, it was demonstrated by several different authors that CSPG degradation with a specific enzyme, chondroitinase ABC, promotes CNS recovery (Bradbury et al., 2002). Previous studies in the laboratory of the present inventors have shown that degradation products of CSPG generated by its degradation with this specific enzyme, chondroitinase ABC, are actually highly potent compounds (see Example 1; Rolls et al., 2004). The degradation products that were studied are the smallest unit of the GAG chain, disaccharides. In studies that were performed in a laboratory of the present inventors, a specific disaccharide of CSPG that is sulfated on the 6-sulfate of the N-acetyl galactosamine was the most active compound. This CSPG-DS as the present inventors have previously shown endows neurons with the ability to withstand threatening conditions regardless of the toxic factor, via activation of an intracellular signaling pathway associated with survival such as PYK2 and PKC. CSPG-DS can promote axonal growth and moreover, it activates microglia towards a neuroprotective phenotype. Actually, CSPG-DS shapes the local innate response of microglia (Example 1; Rolls et al., 2004).

Therefore, since CSPG seems to be associated with glaucoma and since glaucoma is currently considered as a neurodegenerative disorder, based on the previous findings on the potency of the degradation products of CSPG by the present inventors, the present inventors expect that CSPG-DS would be protective in the rat model of glaucoma via a direct effect on neurons and further by activating microglia to a neuroprotective phenotype.

The results presented in the study below indicate that CSPG-DS is highly protective in the rat model of laser-induced glaucoma, both systemically and even more interestingly in an eye-drop formulation.

Materials and Methods

Animals: Inbred adult male Lewis rats (8 weeks; average weight 300 g) were supplied by the Animal Breeding Center at The Weizmann Institute of Science. The rats were maintained in a light- and temperature-controlled room and were matched for age and weight before each experiment. All animals were handled according to the regulations formulated by IACUC (International Animal Care and Use Committee).

Induction of chronically high intra-ocular pressure: Blockage of aqueous outflow causes an increase in IOP, which results in RGC death (Schori et al., 2001 and Bakalash et al., 2002). An increase in IOP was achieved in the right eyes of deeply anesthetized rats (ketamine hydrochloride 50 mg/kg, xylazine hydrochloride 0.5 mg/kg, injected intramuscularly) by blocking the aqueous outflow in that eye with 80-120 applications of blue-green argon laser radiation from a Haag-Streit slit lamp. The laser beam, which was directed at three of the four episcleral veins and at 270 degrees of the limbal plexus, was applied with a power of 1 watt for 0.2 s, producing a spot size of 100 mm at the episcleral veins and 50 mm at the limbal plexus. At a second laser session 1 week later, the same parameters were used except that the spot size was 100 mm for all applications, this time the radiation was directed towards all four episcleral veins and 360 degrees of the limbal plexus (Schori et al., 2001).

Measurement of intraocular pressure: Most anesthetic agents cause a reduction in IOP (Jia et al., 2000), thus precluding reliable measurement. To obtain accurate pressure measurements while the rat was in a tranquil state, the rat was injected intraperitoneally (i.p.) with acepromazine 10 mg/ml and measured the pressure in both eyes 5 minutes later using a Tono-Pen XL tonometer (Automated Ophthalmics, Ellicott City, Md., USA), after applying Localin to the cornea. Because of the reported effect of anesthetic drugs on IOP measured by Tono-Pen (Jia et al., 2000), measurement was always made at the same time after acepromazine injection and the average of 10 values received from each eye was recorded. Measurements were performed every 2 days for 3 weeks, all at the same time of day.

Anatomical assessment of retinal damage caused by the increase in IOP: The hydrophilic neurotracer dye dextran tetramethylrhodamine (Rhodamine Dextran) (Molecular Probes, Oreg., USA) was applied directly into the intra-orbital portion of the optic nerve. Only axons that survive the high IOP and remain functional, and whose cell bodies are still viable, can take up the dye and demonstrate labeled RGCs. The rats were euthanized 24 hours after dye application and their retinas were excised, whole-mounted, and preserved in 4% paraformaldehyde. RGCs were counted under magnification of ×800 in a Zeiss fluorescent microscope. Four fields from each retina were counted, all with the same diameter (0.076 mm²) and located at the same distance from the optic disc (Kipnis et al., 2001; and Yoles et al., 2001). Eyes from untreated rats were used as a control. RGCs were counted by an observer who was blinded to the identity of the retinas.

CSPG-DS administration: CSPG-DS was dissolved in PBS (Sigma-Aldrich, St. Louis, Mo.) and given at different concentrations and at different time points after the primary insult subcutaneously. Topical administration of CSPG-DS was done after immersing the substance in PBS at a concentration of 20 μg/ml. Since each drop was of 50 microliter, 1 drop was administered every 5 minutes for a total of 5 drops in 25 minutes.

Results

CSPG-DS Reduces Death of RGCs Exposed to Chronic Elevation of IOP.

Glaucoma is considered as a neurodegenerative disorder caused by high intra ocular pressure (IOP). Two different models simulate the death induced by either chronic or acute IOP elevation. Death kinetics differ markedly between these two models due to the nature of the primary insult. In the in vivo model of chronic glaucoma used in the laboratory of the present inventors, it was induced by blockage of aqueous outflow from the eye in two sessions of argon laser, which cause an increase in IOP and results in RGC death (Schori et al., 2001; and Bakalash et al., 2002). To examine the effects of CSPG-DS on neuronal survival in this model, CSPG-DS (15 μg/rat) was administered intravenously in several regimens. The regimens were adopted from previous studies on this model (Schori et al., 2001; and Bakalash et al., 2002), which indicated that there was no effect for treatment prior to day 7 after the first laser session. Therefore, the first group of animals was injected with CSPG-DS (15 g/rat) seven days after the first laser session; the second group of animals was injected every other day between day 7 and day 14 with 15g/ml of CSPG-DS at each injection. The later regimen was the effective one, yielding survival of 2063±215 RGCs per mm² (n=5) as compared to the PBS injected group (n=7) where the number of viable RGCs was 1424±236 (p<0.001) (FIG. 11). CSPG-DS induces neurporotection when given as eye drops

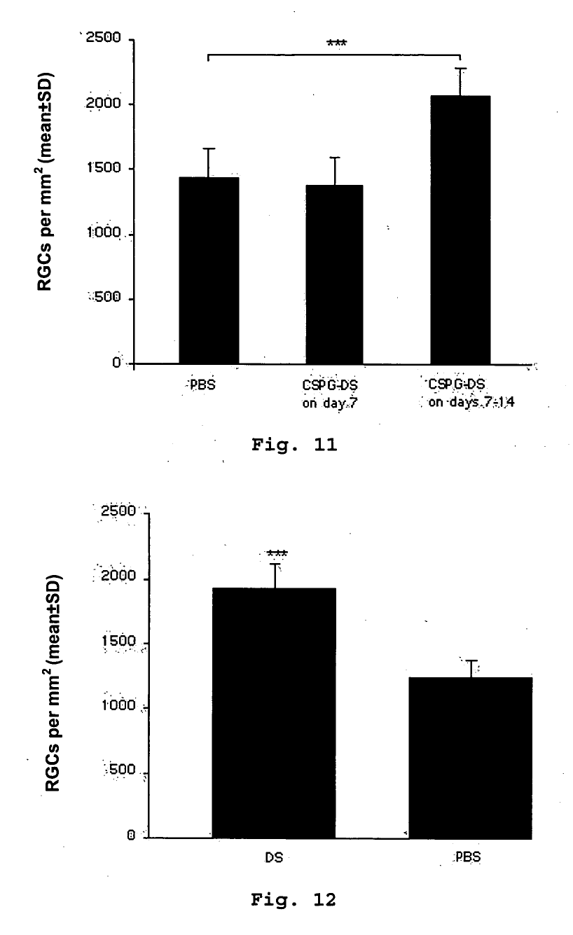

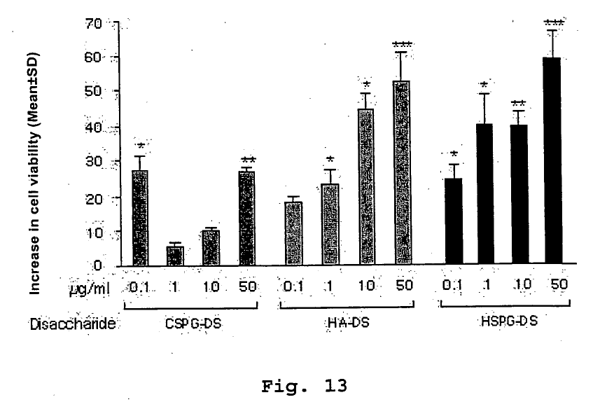

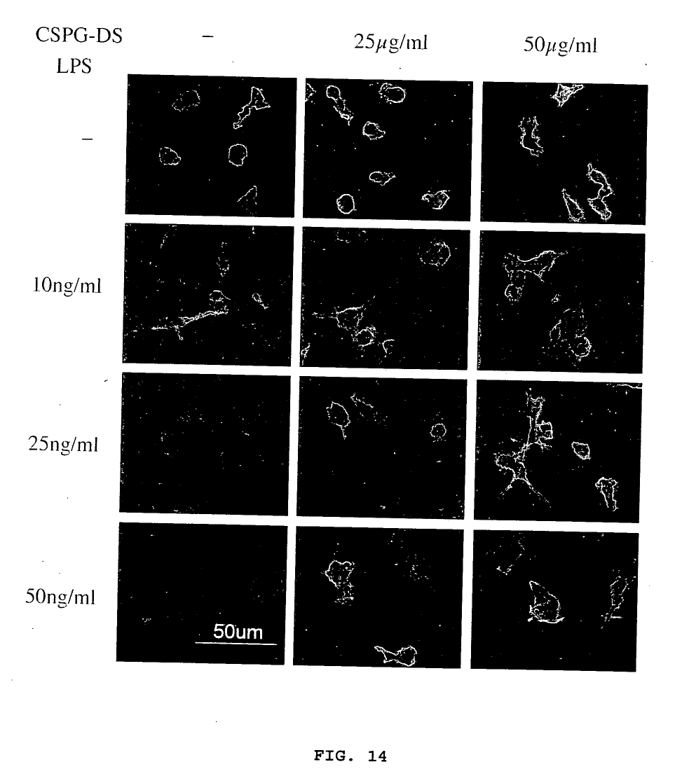

Based on the observed effect of CSPG-DS on neuronal survival in the in vivo model of chronic glaucoma used when CSPG-DS were introduced systemically, the present inventors hypothesized that CSPG-DS being a very low molecular weight compound (600 Dalton) if injected as an eye drop, can penetrate the cornea and eventually reach the RGC layer to induce a direct effect on cell body protection from the outcome of increased IOP. To test this hypothesis, CSPG-DS was applied as eye drops onto the cornea of eyes subjected to chronic elevation of IOP (FIG. 12). The frequency of administration from the previous experiment was used and CSPG-DS was topically applied every other day between day 7 and day 14. Retinas were labeled, excised and counted for viable RGCs three weeks after the first laser irradiation. CSPG-DS treated animals (1924±191 RGCs per mm²; n=6) exhibited significantly higher cell numbers per mm² than the control (PBS-treated) group (1229±146 per mm²; n=4; p<0.001).

### EXAMPLE 4

### Disaccharides Derived From Various Sources Can Promote Neuronal Survival

Disaccharides (DS) can be derived from various sources including proteoglycans. Here, DS from chondroitin sulfate proteoglycan (CSPG-DS), previously shown as neuroprotective, as well as DS from heparan sulfate proteoglycan (HSPG) and from hyaluronic acid (HA) are examined.

Materials and Methods

Reagents. Horse serum, FCS, antibiotics, sodium pyruvate, and DMEM were from Beit-Ha-Emek (Kibbutz Beit Ha-Emek; Israel). Nerve growth factor (NGF) and the XTT viability kit were from Sigma-Aldrich (St. Louis,. Mo.). Collagen was purchased from Calbiochem Novabiochem (Darmstadt, Germany). The 6-sulfated sodium salt (Di-6S) of CSPG-DS (C4170), were purchased from Sigma (Steinheim, Germany).

PC12 cell line. Rat pheochromocytoma (PC12) cells were cultured in DMEM containing horse serum and FCS, both at 8% (culture medium) or at 1% (differentiation medium).

Cell viability assay. PC12 cells were seeded on collagen-coated 96-well plates at a density of 10⁴ cells per well (in differentiation medium containing 100 ng/ml NGF). The cells were incubated with CSPG-DS or other disaccharides at the indicated concentrations for 45 min, then washed with PBS and exposed to glutamate (10⁻³ M) for 15 min. The glutamate solution was washed away and replaced with DMEM for a further 24 h of incubation. The number of viable cells was then determined with the XTT viability kit according to the manufacturer's instructions.

Results

CSPG-DS as well as the other disaccharides examined in this assay protected PC12 cells from glutamate toxicity. In FIG. 13, survival of PC12 cells in the presence of glutamate increased with increasing doses of added disaccharides (between 1 and 50 μg/ml). The disaccharides derived from hyaluronic acid (HA) as well as those derived from heparan sulfate (HSPG), were efficient in promoting neuronal survival, which indicates a general feature of disaccharides regardless of their source.

### EXAMPLE 5

### CSPG-DS in Promoting Neurogenesis

The functional recovery from various neurodegenerative diseases as well as following acute CNS or PNS insults is determined by effective axonal regeneration, survival of spared neurons (either directly or through the glia supportive tissue) or replacement of dead cells by new cells (neurons or glia), a process termed as neurogenesis. In previous studies, the laboratory of the present inventors has demonstrated that CSPG-DS as well as other disaccharides and proteoglycan degradation products can promote recovery of the CNS. The possible mechanism by which CSPG-DS may induce its beneficial effects in the injured CNS were examined. These studies indicated that these effects arise from the direct activation of neurons (inducing axonal growth and promoting cell survival) and the effects on other support systems such as activation of microglia and astrocytes as well as modulation of the T cell mediated responses, thus resulting in better survival of the cells in the injured environment.

Further investigation of the CSPG-DS effects on microglia by the present inventors has led to the finding that CSPG-DS induces microglia to express high levels of IGF-1. Lippopolysaccharides (LPS) application on microglia is known to suppress IGF-I expression from these cells, however pre-incubation of microglia with CSPG-DS attenuated the LPS-induced suppression (FIG. 14). Therefore, treatment with CSPG-DS (and oligosaccharide degradation products) can promote the formation of new neural cells.

Materials and Methods

Primary microglial culture. Brains from neonatal (P0-P1) C57Bl/6J mice were stripped of their meninges and minced with scissors under a dissecting microscope (Zeiss, Stemi DV4, Germany) in Leibovitz-15 medium (Biological Industries, Beit Ha-Emek, Israel). After trypsinization (0.5% trypsin, 10 min, 37° C./5% CO₂), the tissue was triturated. The cell suspension was washed in culture medium for glial cells (DMEM supplemented with 10% fetal calf serum (FCS; Sigma-Aldrich, Rehovot, Israel), 1-glutamine (1 mM), sodium pyruvate (1 mM), penicillin (100 U/ml), and streptomycin (100 mg/ml)) and cultured at 37° C./5% CO₂ in 75-cm₂ Falcon tissue-culture flasks (BD Biosciences) coated with poly-D-lysine (PDL) (10 mg/ml; Sigma-Aldrich, Rehovot) in borate buffer (2.37 g borax and 1.55 g boric acid dissolved in 500 ml sterile water, pH 8.4) for 1 h, then rinsed thoroughly with sterile, glass-distilled water. Half of the medium was changed after 6 h in culture and every 2nd day thereafter, starting on day 2, for a total culture time of 10-14 days. Microglia were shaken off the primary mixed brain glial cell cultures (150 rpm, 37° C., 6 h) with maximum yields between days 10 and 14, seeded (105 cells/ml) onto PDL-pretreated 24-well plates (1 ml/well; Corning, Corning, N.Y.), and grown in culture medium for microglia (RPMI-1640 medium (Sigma-Aldrich, Rehovot) supplemented with 10% FCS, 1-glutamine (1 mM), sodium pyruvate (1 mM), β-mercapto-ethanol (50 mM), penicillin (100 U/ml), and streptomycin (100 mg/ml)). The cells were allowed to adhere to the surface of a PDL-coated culture flask (1 h, 37° C./5% CO₂), and non-adherent cells were rinsed off.

CSPG-DS. disaccharides were purchased from Sigma (C-4170).

Immunocytochemistry. Cover slips from microglia cultures were washed with PBS and fixed with PFA 4%. The covers were treated with a permeabilization/blocking solution containing 10% FCS, 2% bovine serum albumin, 1% glycine, and 0.1% Triton X-100 (Sigma-Aldrich) and stained with goat anti-IGF-I (1:2 0 dilution; R&D Systems).

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Anderson et al. (2002). “Insulin-like growth factor-I and neurogenesis in the adult mammalian brain.” Brain Res Dev Brain Res 134(1-2):115-22
Arakawa Y, B. H., Furuyashiki T, Tsuji T, Takemoto-Kimura S, Kimura K, Nozaki K, Hashimoto N, Narumiya S. (2003) Control of axon elongation via an SDF-1alpha/Rho/mDia pathway in cultured cerebellar granule neurons. J Cell Biol 161, 381-391
Ariel, A., Yavin, E. J., Hershkoviz, R., Avron, A., Franitza, S., Hardan, I., Cahalon, L., Fridkin, M., and Lider, O. (1998) IL-2 induces T cell adherence to extracellular matrix: inhibition of adherence and migration by IL-2 peptides generated by leukocyte elastase. J. Immunol. 161:2465-2472.
Bakalash, S., Kipnis, J., Yoles, E., and Schwartz, M. (2002) Resistance of retinal ganglion cells to an increase in intraocular pressure is immune-dependent. Invest. Ophthalmol. Vis. Sci. 43:2648-2653.
Bakalash, S., Mizrahi, T., Kessler, A., Nussenblatt, R., and Schwartz, M. (2003) Antigenic specificity of immunoprotective therapeutic vaccination for glaucoma. Invest. Opthalmol. Vis. Sci.: In press.
Bettelli, E., Sullivan, B., Szabo, S., Sobel, R., Glimcher, L., and Kuchroo, V. (2004) Loss of T-bet, But Not STAT1, Prevents the Development of Experimental Autoimmune Encephalomyelitis. J. Exp. Med. 200:79-87.
Bradbury E J, M L, Popat R J, King V R, Bennett G S, Patel P N, Fawcett J W, McMahon S B. (2002) Chondroitinase ABC promotes functional recovery after spinal cord injury. Nature 416:636-640.
Bradbury, E. J., Moon, L. D., Popat, R. J., King, V. R., Bennett, G. S., Patel, P. N., Fawcett, J. W., and McMahon, S. B. (2002) Chondroitinase ABC promotes functional recovery after spinal cord injury. Nature 416:636-640.
Bray, et al., (1991) “Neuronal and Nonneuronal Influences on Retinal Ganglion Cell Survival, Axonal Regrowth, and Connectivity After Axotomy”, Ann. N.Y. Acad. Sci.: 214-228
Cadelli, et al., (1992) Exp. Neurol. 115:189-192
Caspi, R. (1999) Immune mechanisms in uveitis. Springer Semin. Immunopathol. 21:113.
Chau, C., Shum, D., Li, H., Pei, J., Lui, Y., Wirthlin, L., Chan, Y., and Xu, X. (2004) Chondroitinase ABC enhances axonal regrowth through Schwann cell-seeded guidance channels after spinal cord injury. FASEB J. 18: 194-196.
Chen D F, J S, Schneider G E. (1995) Intrinsic changes in developing retinal neurons result in regenerative failure of their axons. Proc Natl Acad Sci U S A 92:7287-7291.
Condic, M., Snow, D., and Letourneau, P. (1999) Embryonic neurons adapt to the inhibitory proteoglycan aggrecan by increasing integrin expression. J. Neurosci. 19:10036-10043.
Cooney, R. (2002) Suppressors of cytokine signaling (SOCS): inhibitors of the JAK/STAT pathway. Shock. 17:83-90.
DeWitt, D., and Silver, J. (1996) Regenerative failure: a potential mechanism for neuritic dystrophy in Alzheimer's disease. Exp. Neurol. 142:103-110.
Dickson, B. (2001) Rho GTPases in growth cone guidance. Curr Opin Neurobiol, 103-110
Dillon, G., Yu, X., and Bellamkonda, R. (2000) The polarity and magnitude of ambient charge influences three-dimensional neurite extension from DRGS. J. Biomed. Mater. Res. 51: 510-519.
Dou, C., and Levine, J. (1997) Identification of a neuronal cell surface receptor for a growth inhibitory chondroitin sulfate proteoglycan (NG2). J. Neurochem. 68:1021-1030.
Du et al., (2002) Determination of the chondroitin sulfate disaccharides in dog and horse plasma by HPLC using chondroitinase digestion, precolumn derivatization, and fluorescence detection, Analytical Biochemistry 306:252-258
Ernst, H., Zanin, M., Everman, D., and Hoffman, S. (1995) Receptor-mediated adhesive and anti-adhesive functions of chondroitin sulfate proteoglycan preparations from embryonic chicken brain. J. Cell Sci. 108.
Eyupoglu IY, S. N., Brauer AU, Nitsch R, Heimrich B. (2003) Identification of neuronal cell death in a model of degeneration in the hippocampus. Brain Res Protoc. 11, 1-8
Fang, I., Yang, C., Lin, C., Yang, C., and Chen, M. (2004) Expression of chemokine and receptors in Lewis rats with experimental autoimmune anterior uveitis. Exp. Eye Res. 78:1043-1055.
Ferguson et al., (2000) MMP-2 and MMP-9 increase the neurite-promoting potential of schwann cell basal laminae and are upregulated in degenerated nerve, Molecular and Cellular Neuroscience 16:157-167
Fidler P S, S K, Asher R A, Dobbertin A, Thornton S R, Calle-Patino Y, Muir E, Levine J M, Geller H M, Rogers J H, Faissner A,Fawcett J W. (1999) Comparing astrocytic cell lines that are inhibitory or permissivefor axon growth: the major axon-inhibitory proteoglycan is NG2. J Neurosci 19:8778-8788.
Fitch M T, D. C., Combs CK , Landreth G E, Silver J. (1999) Cellular and molecular mechanisms of glial scarring and progressive cavitation: in vivo and in vitro analysis of inflammation-induced secondary injury after CNS trauma. J. Neurosci. 19, 8182-8198
Fitch M T, S J (1997) Activated macrophages and the blood-brain barrier: inflammation after CNS injury leads to increases in putative inhibitory molecules. Exp Neurol 148:587-603.
Franklin K B J, Paxinos G (1997) In: The Mouse Brain in Stereotaxic Coordinates, Academic Press, San Diego
Fukuda, T., Kawano, H., Ohyama, K., Li, H., Takeda, Y., Oohira, A., and Kawamura, K. (1997) Immunohistochemical localization of neurocan and Li in the formation of thalamocortical pathway of developing rats. J. Comp. Neurol. 382:141-152.
Ghosh, S., May, M. J., and Kopp, E. B. (1998) NF-{kappa} B and Rel proteins: evolutionarily conserved mediators of immune responses. Annu. Rev. Immunol. 16:225-260.
Grimpe B, S J (2002) The extracellular matrix in axon regeneration. Prog Brain Res 137:333-349.
Hascall, V., and Sajdera, S. (1970) Physical properties and polydispersity of proteoglycan from bovine nasal cartilage. J. Biol. Chem. 245: 4920-4930.
Hauben et al, (2000) “Autoimmune T cells as potential neuroprotective therapy for spinal cord injury”, Lancet 355:286-287
Huang et al., (1995) Determination of chondroitin sulphates in human whole blood, plasma and blood cells by high-performance liquid chromatography, Biomedical Chromatography 9:102-105
Jia, L., Cepurna, W., Johnson, E., and Morrison, J. (2000) Patterns of intraocular pressure elevation after aqueous humor outflow obstruction in rats. Invest. Ophthalmol. Vis. Sci. 41:1380-1385.
Johnson, E., Morrison, J., Farrell, S., Deppmeier, L., Moore, C., and McGinty, M. (1996) The effect of chronically elevated intraocular pressure on the rat optic nerve head extracellular matrix. Exp. Eye Res. 62:663-674.
Johnson, H., Torres, B., and Soos, J. (1996) Superantigens: structure and relevance to human disease. Proc. Soc. Exp. Biol. Med. 212:99-109.
Jones L L, Y Y, Stallcup W B, Tuszynski M H. (2002) NG2 is a major chondroitin sulfate proteoglycan produced after spinal cord injury and is expressed by macrophages and oligodendrocyte progenitors. J Neurosci 22:2792-2803.
Jones, L. L., Yamaguchi, Y., Stallcup, W. B., and Tuszynski, M. H. (2002) NG2 is a major chondroitin sulfate proteoglycan produced after spinal cord injury and is expressed by macrophages and oligodendrocyte progenitors. J. Neurosci. 22:2792-2803.
Kipnis, J., Yoles, E., Schori, H., Hauben, E., Shaked, I., and Schwartz, M. (2001) Neuronal survival after CNS insult is determined by a genetically encoded autoimmune response. J. Neurosci. 21:4564-4571.
Kitagawa, H., Tsutsumi, K., Tone, Y., and Sugahara, K. (1997) Developmental regulation of the sulfation profile of chondroitin sulfate chains in the chicken embryo brain. J. Biol. Chem. 272:31377-31381.
Knepper, P., Goossens, W., Hvizd, M., and Palmberg, P. (1996) Glycosaminoglycans of the human trabecular meshwork in primary open-angle glaucoma. Invest. Ophthalmol. Vis. Sci. 37:1360-1367.
Kobayashi, K., Kaneda, K., and Kasama, T. (2001) Immunopathogenesis of delayed-type hypersensitivity. Microsc. Res. Tech. 53:241-245.
Koga, T., Inatani, M., Hirata, A., Inomata, Y., Oohira, A., Gotoh, T., Mori, M., and Tanihara, H. (2003) Expression of glycosaminoglycans during development of the rat retina. Curr. Eye Res. 27:75-83..
Kranenburg O, P. M., van Horck F P, Drechsel D, Hall A, Moolenaar W H. (1999) Activation of RhoA by lysophosphatidic acid and Galphal2/13 subunits in neuronal cells: induction of neurite retraction. Mol Biol Cell 10, 1851-1857
Lehman, K., (1971) Acrylic Coatings in Controlled Realse Tablet Manufacturer, Manufacturing Chemist and Aerosol News, p. 39
Lemons, M., Howland, D., and Anderson, D. (1999) Chondroitin sulfate proteoglycan immunoreactivity increases following spinal cord injury and transplantation. Exp. Neurol. 160: 51-65.
Lips, K., Stichel, C., and Muller, H. (1995) Restricted appearance of tenascin and chondroitin sulphate proteoglycans after transection and sprouting of adult rat postcommissural fornix. J. Neurocytol. 24:449-464.
Lublin, F. (1985) Relapsing experimental allergic encephalomyelitis. An autoimmune model of multiple sclerosis. Springer Semin. Immunopathol. 8:197-208.
Makarov, S. (2000) NF-{kappa}B as a therapeutic target in chronic inflammation: recent advances. Mol. Med. Today 6:441-448.
Matsui, F., Kawashima, S., Shuo, T., Yamauchi, S., Tokita, Y., Aono, S., Keino, H., and Oohira, A. (2002) Transient expression of juvenile-type neurocan by reactive astrocytes in adult rat brains injured by kainate-induced seizures as well as surgical incision. Neuroscience 112:773-781.
McKeon R J, H A, Silver J. (1995) Injury-induced proteoglycans inhibit the potential for laminin-mediated axon growth on astrocytic scars. Exp Neurol 136:32-43.
McKeon, R., Jurynec, M., and Buck, C. (1999) The chondroitin sulfate proteoglycans neurocan and phosphacan are expressed by reactive astrocytes in the chronic CNS glial scar. J.
Neurosci. 19:10778-10788.
Meyer-Puttlitz, B., Junker, E., Margolis, R., and Margolis, R. (1996) Chondroitin sulfate proteoglycans in the developing central nervous system. II. Immunocytochemical localization of neurocan and phosphacan. J. Comp. Neurol. 366:44-54.
Mizrahi T., H., E. Schwartz, M. (2002) The tissue-specific self-pathogen is the protective self-antigen: The case of uveitis. J. Immunol. 169, 5971-5977
Moalem et al., (1999b) Differential T cell response in central and peripheral nerve injury: connection with immune privilege, Faseb J, 13:1207-17
Moalem, G, Leibowitz-Amit, R, Yoles, E, Mor, F, Cohen, I R, Schwartz, M (1999) Autoimmune T cells protect neurons from secondary degeneration after central nervous system axotomy. Nat Med 5:49-55.
Moalem, G, Yoles, E, Leibowitz-Amit, R, Muller-Gilor, S, Mor, F, Cohen, I R, Schwartz, M (2000) Autoimmune T cells retard the loss of function in injured rat optic nerves. J Neuroimmunol 106:189-197.
Monnier P P, S. A., Schwab JM, Henke-Fahle S, Mueller B K. (2003) The Rho/ROCK pathway mediates neurite growth-inhibitory activity associated with the chondroitin sulfate proteoglycans of the CNS glial scar. Mol Cell Neurosci 22, 319-330
Morgenstern, D., Asher, R., and Fawcett, J. (2002) Chondroitin sulphate proteoglycans in the CNS injury response. Prog. Brain Res. 137:313-332.
Morris, J. (1979) Steric exclusion of cells. A mechanism of glycosaminoglycan-induced cell aggregation. Exp. Cell Res. 120:141-153.
Moser, B., Loetscher, M., Piali, L., and Loetscher, P. (1998) Lymphocyte responses to chemokines. Int. Rev. Immunol. 16:323-344.
Nevo, U, Kipnis, J, Golding, I, Shaked, I, Neumann, A, Akselrod, S, Schwartz, M (2003) Autoimmunity as a special case of immunity: removing threats from within. Trends Mol Med 9:88-93.
Niederost B, O. T., Fritsche J, McKinney R A, Bandtlow C E. (2002) Nogo-A and myelin-associated glycoprotein mediate neurite growth inhibition by antagonistic regulation of RhoA and Racl. J Neurosci 22, 10368-10376.
Olsson, T. (1995) Critical influences of the cytokine orchestration on the outcome of myelin antigen-specific T-cell autoimmunity in experimental autoimmune encephalomyelitis and multiple sclerosis. Immunol. Rev. 144:245-268.
Pashenkov, M., Soderstrom, M., and Link, H. (2003) Secondary lymphoid organ chemokines are elevated in the cerebrospinal fluid during central nervous system inflammation. J. Neuroimmunol. 135:154-160.
Popovich, P. G., Stokes, B. T. & Whitacre, C. C. (1996) Concept of autoimmunity following spinal cord injury: possible roles for T lymphocytes in the traumatized central nervous system. J Neurosci Res 45, 349-363
Properzi, F., Asher, R., and Fawcett, J. (2003) Chondroitin sulphate proteoglycans in the central nervous system: changes and synthesis after injury. Biochem. Soc. Trans. 31:335-336.
Rolls, A. H., Avidan., Liora, C., Hadas, S., Sharon, B., Vladimir, L., Sima, L., Ofer, L., and Michal, S. (2004) A Disaccharide Derived from Chondroitin Sulfate Proteoglycan Promotes Central Nervous System Repair. Eur. J. Neurosci.
Saito et al., (1968) Enzymatic methods for the determination of small quantities of isomeric chondroitin sulfates, The Journal of Biological Chemistry 243(7)1536-1542
Schori, H., Kipnis, J., Yoles, E., WoldeMussie, E., Ruiz, G., Wheeler, L.A., and Schwartz, M. (2001) Vaccination for protection of retinal ganglion cells against death from glutamate cytotoxicity and ocular hypertension: implications for glaucoma. Proc. Natl. Acad. Sci. U S A 98:3398-3403.
Schori, H., Yoles, E. & Schwartz, M. (2001) T-cell-based immunity counteracts the potential toxicity of glutamate in the central nervous system. J. Neuroimmunol. 119, 199-204
Schori, H., Yoles, E., Wheeler, L. A. & Schwartz, M. (2002) Immune related mechanisms participating in resistance and susceptibility to glutamate toxicity. Eur. J. Neurosci. 16, 557-564
Schwab, (1991) Phil. Trans. R. Soc. Lond. 331: 303-306
Schwartz, M (2001) T cell mediated neuroprotection is a physiological response to central nervous system insults. J Mol Med 78:594-597.
Schwartz, M (2003) Macrophages and microglia in central nervous system injury: are they helpful or harmful? J Cereb Blood Flow Metab 23:385-394.
Schwartz, M., Cohen, I., Lazarov-Spiegler, O., Moalem, G. & Yoles, E. (1999) The remedy may lie in ourselves: prospects for immune cell therapy in central nervous system protection and repair [In Process Citation]. J. Mol. Med. 77, 713-717
Silver, J., and Miller, J. (2004) Regeneration beyond the glial scar. Nat. Rev. Neurosci. 5:146-156.
Sobel, R., and Ahmed, A. (2001) White matter extracellular matrix chondroitin sulfate/dermatan sulfate proteoglycans in multiple sclerosis. J. Neuropathol. Exp. Neurol. 60:1198-1207.
Soriano, S., Hernanz-Falcon, P., Rodriguez-Frade, J., De Ana, A., Garzon, R., Carvalho-Pinto, C., Vila-Coro, A., Zaballos, A., Balomenos, D., Martinez-A, C., et al. (2002) Functional inactivation of CXC chemokine receptor 4-mediated responses through SOCS3 up-regulation. J. Exp. Med. 196:311-321.
Sugahara et al., (1996) Structural analysis of unsaturated hexasaccharides isolated from shark cartilage chondroitin sulfate D are substrates for the exolytic action of chondroitin ABC lyase, Eur. J. Biochem. 239:871-876
Tang, X., Davies, J. E., and Davies, S. J. (2003) Changes in distribution, cell associations, and protein expression levels of NG2, neurocan, phosphacan, brevican, versican V2, and tenascin-C during acute to chronic maturation of spinal cord scar tissue. J. Neurosci. Res. 71:427-444.
Tanihara, H., Inatani, M., Koga, T., Yano, T., and Kimura, A. (2002) Proteoglycans in the eye. Cornea. 21(7 Suppl):S62-69.
Tatagiba M, B. C., Schwab M E. (1997) Regeneration of injured axons in the adult mammalian central nervous system. Neurosurgery 40, 541-546
Tezel, G., Edward, D., and Wax, M. (1999) Serum autoantibodies to optic nerve head glycosaminoglycans in patients with glaucoma. Arch. Ophthalmol. 117:917-924.
Thurau, S., and Wildner, G. (2003) An HLA-peptide mimics organ-specific antigen in autoimmune uveitis: its role in pathogenesis and therapeutic induction of oral tolerance. Autoimmun. Rev. 2:171-176.
Tigyi G, F. D., Sebok A, Marshall F, Dyer DL, Miledi R. (1996) Lysophosphatidic acid-induced neurite retraction in PC12 cells: neurite-protective effects of cyclic AMP signaling. J Neurochem 66, 549-558
Tropea, D., Caleo, M., and Maffei, L. (2003) Synergistic effects of brain-derived neurotrophic factor and chondroitinase ABC on retinal fiber sprouting after denervation of the superior colliculus in adult rats. J. Neurosci. 23:7034-7044.
Vaday G G, L O (2000) Extracellular matrix moieties, cytokines, and enzymes: dynamic effects on immune cell behavior and inflammation. J Leukoc Biol 67:149-159.
Volpi, (2000) Hyaluronic acid and chondroitin sulfate unsaturated disaccharides analysis by high-performance liquid chromatography and fluorimetric detection with dansylhydranzine, Analytical Biochemistry 277:19-24
Werb, (1997) ECM and cell surface proteolysis: regulating cellular ecology, Cell 91:439-442
Wilson, M., and Snow, D. (2000) Chondroitin sulfate proteoglycan expression pattern in hippocampal development: potential regulation of axon tract formation. J. Comp. Neurol. 424:532-546.
Wu, D., Woodman, S., Weiss, J., McManus, C., D'Aversa, T., Hesselgesser, J., Major, E., Nath, A., and Berman, J. (2000) Mechanisms of leukocyte trafficking into the CNS. J. Neurovirol. 6:S82-85.
Yick, L. W., Wu, W., So, K. F., Yip, H. K., and Shum, D. K. (2000) Chondroitinase ABC promotes axonal regeneration of Clarke's neurons after spinal cord injury. Neuroreport 11:1063-1067.
Yoles, E, Schwartz, M (1998) Elevation of intraocular glutamate levels in rats with partial lesion of the optic nerve. Arch Ophthalmol 116:906-910.
Yoles, E., Hauben, E., Palgi, O., Agranov, E., Gothilf, A., Cohen, A., Kuchroo, V., Cohen, I. R., Weiner, H., and Schwartz, M. (2001) Protective autoimmunity is a physiological response to CNS trauma. J. Neurosci. 21:3740-3748.
Zuo, J., Hernandez, Y., and Muir, D. (1998) Chondroitin sulfate proteoglycan with neurite-inhibiting activity is up-regulated following peripheral nerve injury. J. Neurobiol. 34:41-54.
Zuo, J., Neubauer, D., Graham, J., Krekoski, C. A., Ferguson, T. A., and Muir, D. (2002) Regeneration of axons after nerve transection repair is enhanced by degradation of chondroitin sulfate proteoglycan. Exp. Neurol. 176:221-228.

What is claimed is:

1. A method for treating, inhibiting, or ameliorating the effects of injuries or diseases that result in neuronal degeneration or the effects of disorders that result in mental or cognitive dysfunction, comprising administering to a patient in need thereof an effective amount of at least one oligosaccharide or an amount of activated microglial cells, stem cells or neuronal progenitor cells which have been treated with an effective amount of at least one oligosaccharide prior to being administered by implantation at the site of neuronal degeneration.

2. The method of claim 1, wherein the at least one oligosaccharide is a degradation product of a naturally-occurring proteoglycan.

3. The method of claim 2, wherein the naturally-occurring proteoglycan is a human proteoglycan.

4. The method of claim 2, wherein the naturally-occurring proteoglycan is a chondroitin sulfate proteoglycan.

5. The method of claim 2, wherein the at least one oligosaccharide is an enzymatic degradation product of a chondroitin sulfate proteoglycan.

6. The method of claim 1, wherein the at least one oligosaccharide is a sulfated oligosaccharide.

7. The method of claim 1, wherein the at least one oligosaccharide comprises a disaccharide.

8. The method of claim 7, wherein the disaccharide is a degradation product of a naturally-occurring proteoglycan.

9. The method of claim 8, wherein the naturally-occurring proteoglycan is a chondroitin sulfate proteoglycan.

10. The method of claim 8, wherein the naturally-occurring proteoglycan is a heparan sulfate proteoglycan.

11. The method of claim 8, wherein the naturally-occurring proteoglycan is hyaluronic acid.

12. The method of claim 7, wherein the disaccharide is a degradation product from a glycosaminoglycan chain of a naturally-occurring proteoglycan.

13. The method of claim 7, wherein the disaccharide is a sulfated disaccharide.

14. The method of claim 13, wherein the sulfated disaccharide is 2-acetamido-2-deoxy-3-O-(β-D-gluco-4-enepyranosyuronic acid)-6-O-sulfo-D-galactose.

15. The method of claim 1, in which the injury, disease or disorder is caused or exacerbated by glutamate toxicity.

16. The method of claim 1, in which the injury, disease or disorder is spinal cord injury, blunt trauma, penetrating trauma, hemorrhagic stroke, or ischemic stroke.

17. The method of claim 1, in which the injury, disease or disorder is a neurodegenerative disease.

18. The method of claim 17, wherein the neurodegenerative disease is glaucoma or Alzheimer's disease.

19. The method of claim 1, in which the injury, disease or disorder results in mental or cognitive dysfunction.

20. The method of claim 19, wherein the mental or cognitive dysfunction is a mental disorder.

21. The method of claim 4, wherein administering an effective amount of at least one oligosaccharide degradation product of a naturally occurring proteoglycan to a patient in need thereof promotes the formation of new neural cells.

22. The method of claim 21, wherein the naturally-occurring proteoglycan is chondroitin sulfate proteoglycan.

23. The method of claim 21, wherein the at least one oligosaccharide comprises a disaccharide degradation product of a naturally occurring proteoglycan.

24. The method of claim 23, wherein the naturally occurring proteoglycan is chondroitin sulfate proteoglycan.

25. A method for promoting neurogenesis, comprising administering to a patient in need thereof an effective amount of at least one oligosaccharide degradation product of a naturally occurring proteoglycan.

26. The method of claim 25, wherein the naturally-occurring proteoglycan is chondroitin sulfate proteoglycan.

27. The method of claim 25, wherein the at least one oligosaccharide comprises a disaccharide degradation product of a naturally occurring proteoglycan.

28. The method of claim 27, wherein the naturally occurring proteoglycan is chondroitin sulfate proteoglycan.
